(19) United States
(12) Patent Application Publication
(10) Pub. No.: US 2008/0279781 A1
(43) Pub. Date: Nov. 13, 2008

(54) Glycosylated Carboranylporphyrins and Uses Thereof

(75) Inventors: Michiko Miura, Hampton Bays, NY (US); Haitao Wu, Rockville, MD (US)

Correspondence Address: BROOKHAVEN SCIENCE ASSOCIATES/;BROOKHAVEN NATIONAL LABORATORY, BLDG. 185 - P.O. BOX 5000, UPTON, NY, 11973, US

(73) Assignee: Brookhaven Science Associates, LLC, Upton, NY (US)

(21) Appl. No.: 11/746,957

(22) Filed: May 10, 2007

(51) Int. Cl. A61K 49/00; A61K 31/40; A61P 43/00; C07D 225/00; C07F 5/00

(52) U.S. Cl. 424/9.35; 514/410; 534/15; 540/465

(57) ABSTRACT

The present invention is directed to low toxicity boronated compounds and methods for their use in the treatment, visualization, and diagnosis of tumors. More specifically, the present invention is directed to low toxicity glycosylated carborane-containing 5,10,15,20-tetraphenylporphyrin compounds and methods for their use particularly in boron neutron capture therapy (BNCT) and photodynamic therapy (PDT) for the treatment of tumors of the brain, and head and neck. The invention is also directed to using these glycosylated carborane-containing tetraphenylporphyrin compounds in methods of tumor imaging and/or diagnosis such as MRI, SPECT, or PET.

This invention was made with Government support under contract number DE-AC02-98CH10886, awarded by the U.S. Department of Energy. The Government has certain rights in the invention.

### BACKGROUND OF THE INVENTION

The efficacy of radiation and chemical methods in the treatment of cancers has been limited by a lack of selective targeting of tumor cells by the therapeutic agent. In an effort to spare normal tissue, current tumor treatment methods have therefore restricted radiation and/or chemical treatment doses to levels that are well below optimal or clinically adequate. Thus, designing compounds that are capable, either alone or as part of a therapeutic method, of selectively targeting and destroying tumor cells, is a field of intense study.

Because of the known affinity of porphyrins to neoplastic tissues, there has been intense interest in using porphyrins as delivery agents in the treatment of neoplasms in brain, head and neck, and related tumors. Porphyrins in general belong to a class of colored, aromatic tetrapyrrole compounds, some of which are found naturally in plants and animals, e.g., chlorophyll and heme, respectively.

Porphyrins and other tetrapyrroles with relatively long singlet lifetimes have already been used to treat malignant tumors using photodynamic therapy (PDT). In PDT, the patient is first injected with a photosensitizing drug, typically a porphyrin. The tumor cells, now photosensitized, are susceptible to destruction when exposed to an intense beam of laser red light. The biochemical mechanism of cell damage in PDT is believed to be mediated largely by singlet oxygen, which is produced by transfer of energy from the light-excited porphyrin molecule to an oxygen molecule. However, PDT has been limited predominantly by the low penetration depth (˜a few millimeters) by visible light.

A promising new form of cancer therapy is boron neutron-capture therapy (BNCT). BNCT is a bimodal cancer treatment based on the selective accumulation of a stable nuclide of boron known as boron-10, or ¹⁰B, in the tumor, followed by irradiation of the tumor with thermalized neutrons. The thermalized neutrons impinge on the boron-10, causing nuclear fission (decay reaction). The nuclear fission reaction causes the highly localized release of vast amounts of energy in the form of high linear-energy-transfer (LET) radiation, which can kill cells more efficiently (higher relative biological effect) than low LET radiation, such as x-rays.

Boron-10 undergoes the following nuclear reaction when capturing a thermal neutron:

¹⁰B+n→¹¹B

¹¹B→⁷Li+⁴He+γ(478 keV)

In this nuclear reaction, a boron-10 nucleus captures a neutron forming the metastable nuclide ¹¹B, which spontaneously and nearly instantaneously disintegrates into a ⁴He and ⁷Li particle, which together possess an average total kinetic energy of 2.34 MeV. These two ionized particles travel about 9 μm and 5 μm (7±2 μm) in opposite directions in soft tissue, respectively.

The distances traveled by the ⁴He and ⁷Li particles are comparable to the diameter of many tumor and tumor-associated cells. Therefore, the efficacy of BNCT resides in the production of highly-localized, high LET ionizing radiation within the tumor. The targeted tumor thus receives a large dose of radiation while sparing surrounding normal tissue.

In the case of brain tumors, after administration of the boron compound, the patient's head is irradiated in the general area of the brain tumor with an incident beam or field of epithermal (0.5 eV-10 keV) neutrons. The neutrons become progressively thermalized (average energy approximately 0.04 eV) as they penetrate deeper into the head. As the neutrons become thermalized, they are more readily captured by the boron-10 concentrated in the tumor cells and/or tumor supporting tissues, since the capture cross section is inversely proportional to the neutron velocity.

In BNCT, the boron-containing compound must be non-toxic or of low toxicity when administered in therapeutically effective amounts, as well as being capable of selectively accumulating in cancerous tissue. Although p-boronophenylalanine (BPA) has the advantage of low chemical toxicity, it accumulates in critical normal tissues at levels that are significantly higher than desirable. In particular, ratios of boron concentration in tumors relative to normal brain and tumors relative to blood are approximately 3:1. Such low specificity limits the maximum dose of BPA to a tumor since the allowable dose to normal tissue is the limiting factor.

A particular class of synthetic porphyrins, known as tetraphenylporphyrins, have been the subject of great interest in the design of new boron carrier compounds for BNCT, mainly due to their ease of synthesis. Tetraphenylporphyrins (TPPs) contain four phenyl groups, typically on the 5, 10, 15, and 20 positions of the porphyrin ring. An advantage of TPPs is their ease of synthesis.

The solubility of TPPs can be controlled by substituents, generally on the phenyl rings. Those TPPs containing sulfonate or carboxylate substituents are water-soluble. However, most of the o-closo-carborane-containing TPPs have high lipophilic properties, which can require high amounts of non-aqueous excipients before administration into animals. High amounts of excipients may reduce the biological effect of the porphyrin by, for example, changing the microlocalization within the tumor cell such that the porphyrin may be bound to membranes instead of homogeneously distributed throughout the cell.

In addition, the use of more hydrophilic substituents such as amide, ester, or urea substituents, although significantly more polar than carbon-carbon linkages, are known to hydrolyze under numerous types of conditions. Such hydrolysis is particularly problematic when such bonds are employed to attach the carboranyl group to the porphyrin molecule, since hydrolysis results in loss of the carboranyl group before reaching the target.

Therefore, there continues to be an effort to reduce the lipophilic behavior of TPPs while not compromising their chemical stability or their biological properties. For example, international Patent Application No. WO 01/85736 by Vicente et al describes the synthesis and use of tetraphenylporphyrin compounds that contain hydrophilic carboranyl substituents. A salient feature of the Vicente compounds is the attachment of the carboranyl group to a phenyl ring by a carbon-carbon linkage. Though the carbon-carbon linkage is not prone to hydrolysis or other chemical attack, the substituent is significantly hydrophobic.

The choice of substituents on the phenyl rings of TPPs may also affect a compound's polarity and ability to target cellular membrane receptors. For instance, porphyrins with saccharide substituents have shown selectivity to tumor cells and have been disclosed and studied as being useful for PDT. Porphyrins with sugar moieties also exhibit good solubility in aqueous solution and increased plasma lifetime.

A porphyrin has the advantage of having the ability to chelate metal ions in its interior. Such chelated porphyrins can additionally function as visualization tools for real-time monitoring of porphyrin concentration for treatment planning and/or as diagnostic agents. For example, when chelated to paramagnetic metal ions, porphyrins may function as contrast agents in magnetic resonance imaging (MRI), and when chelated to radioactive metal ions, porphyrins may function as imaging agents for single photon emission computed tomography (SPECT) or positron emission tomography (PET).

In addition, by using chelated boron-containing porphyrins in BNCT, boron concentration and distribution in and around the tumor and all tissues within the irradiated treatment volume can be accurately and rapidly determined noninvasively before and during the irradiation. Such diagnostic information allows BNCT treatment to be performed more quickly, accurately, and safely, by lowering exposures of epithermal neutrons in regions of tissues known to contain high levels of boron. Moreover, the anticipated use of accelerator-generated neutrons would likely produce a significantly lower flux and hence effect longer irradiation times, so that compounds that have longer tumor retention times would become critical.

Accordingly, there is a need for new compounds, especially boron-containing porphyrins, that have long retention times in tumors that selectively target and destroy tumor cells with minimal damage to normal tissue. In addition, there is a need for more effective methods for the treatment of brain, head and neck, and related tumors, and more particularly, more effective BNCT treatments and boron-delivery compounds used therein.

### SUMMARY OF THE INVENTION

The present invention is directed to low toxicity boronated compounds and methods for their use in the treatment, visualization, and diagnosis of tumors. More specifically, the present invention is directed to low toxicity glycosylated boronated 5,10,15,20-tetraphenylporphyrin compounds and methods for their use particularly in boron neutron capture therapy (BNCT) or photodynamic therapy (PDT) for the treatment of tumors of the brain, and head and neck.

In particular, the present invention is directed to boronated tetraphenylporphyrins of the formula

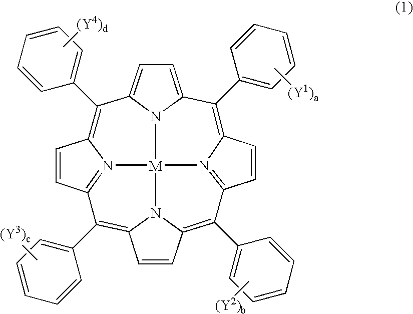

wherein at least one of Y¹, Y², Y³, and Y⁴ represents a monosaccharide, di-saccharide, tri-saccharide, or oligo-saccharide and at least one of Y¹, Y², Y³, or Y⁴ is a substituent represented by formula (2)

—X¹—(CR¹R²)ᵣ-Z (2),

wherein Z is a carborane cluster or a substituent represented by formula (3):

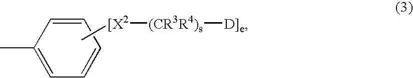

where D is a carborane cluster.

Y¹, Y², Y³, and Y⁴ are independently on either or both of the ortho and/or meta positions or on the para position on the phenyl rings, and a, b, c, and d independently represent 1 or 2.

M is preferably vanadium, manganese, iron, ruthenium, technetium, chromium, platinum, cobalt, nickel, copper, zinc, germanium, indium, tin, yttrium, gold, barium, tungsten, or gadolinium.

In one embodiment, the saccharide component is preferably a mono-saccharide selected from the group consisting of the D or L, alpha and beta forms of glucose, fructose, galactose, mannose, gulose, ribose, arabinose, xylose, and ribulose.

In another embodiment, the saccharide component is preferably a di-saccharide selected from the group consisting of the D or L, alpha and beta forms of sucrose, maltose, and lactose.

In yet another embodiment, a, b, c, and d are 1; two of Y¹, Y², Y³, and Y⁴ are represented by formula (2):

—X¹—(CR¹R²)ᵣ-Z (2);

and the two Y¹-Y⁴ not represented by formula (2) are a mono-saccharide, di-saccharide, tri-saccharide, or oligo-saccharide; and the substituents represented by formula (2) are in the cis configuration on the porphyrin ring.

In yet another embodiment, a, b, c, and d are 1; two of Y¹, Y², Y³, and Y⁴ are the substituents represented by formula (2); the two Y¹-Y⁴ not represented by formula (2) are a mono-saccharide, di-saccharide, tri-saccharide, or oligo-saccharide; and the substituents represented by formula (2) are in the trans configuration on the porphyrin ring.

In yet another embodiment, a, b, c, and d are 1; two of Y¹, Y², Y³, and Y⁴ are the substituents represented by formula (2); X is O; R¹ and R² are H; r is 1; Z is —C₂HB₁₀H₁₀ closo meta-carborane; the two Y¹-Y⁴ not represented by formula (2) are a mono-saccharide, di-saccharide, tri-saccharide, or oligo-saccharide; and the substituents represented by formula (2) are in the trans configuration on the porphyrin ring.

In yet another embodiment, a, b, c, and d are 1; two of Y¹, Y², Y³, and Y⁴ are the substituents represented by formula (2); X is O; R¹ and R² are H; r is 1; Z is the substituent represented by formula (3); X² is O; R³ and R⁴ are H; s is 1; e is 2; D is —C₂HB₁₀H₁₀ closo meta-carborane; the —X²—(CR³R⁴)s-D substituents are in the 3 and 5 positions of each phenyl ring; the two Y¹-Y⁴ not represented by formula (2) are a mono-saccharide, di-saccharide, tri-saccharide, or oligo-saccharide; and the substituents represented by formula (2) are in the trans configuration on the porphyrin ring.

The present invention also includes methods of tumor imaging by SPECT, PET, or MRI, as well as methods of bimodal cancer treatment such as BNCT, XRT, and PDT that require the administration to a subject of a composition that comprises one or more of the porphyrin compounds described above. In a preferred embodiment, the composition is essentially one or more of the porphyrin compounds described above.

### DETAILED DESCRIPTION OF THE INVENTION

The invention relates to glycosylated carboranylporphyrins having the formula

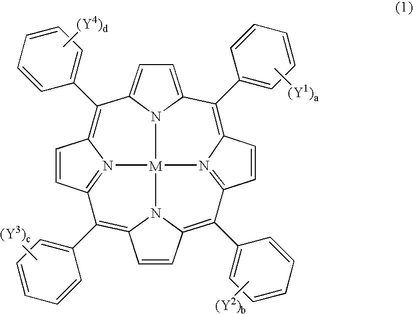

wherein at least one of Y¹, Y², Y³, or Y⁴ represents a mono-saccharide, di-saccharide, tri-saccharide, or oligo-saccharide and at least one of Y¹, Y², Y³, or Y⁴ is a substituent represented by formula (2):

—X¹—(CR¹R²)ᵣ-Z (2),

wherein Z is a carborane cluster or a substituent represented by formula (3):

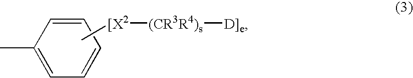

where D is a carborane cluster.

Y¹, Y², Y³, and Y⁴ are independently on either or both of the ortho and/or meta positions or on the para position on the phenyl rings, and a, b, c, and d independently represent 1 or 2.

The Y¹, Y², Y³, or Y⁴ that are not saccharide(s) or substituents represented by formula (2) or formula (3) are independently hydrogen, hydrocarbyl, non-aromatic carbocyclic, non-aromatic heterocyclic, aryl, alkylaryl, arylalkyl, or a hydrocarbyl, non-aromatic carbocyclic, non-aromatic heterocyclic, aryl, alkylaryl, or arylalkyl group substituted with 1 to 4 hydrophilic groups selected from hydroxy, alkoxy, —C(O)OR⁵, —SOR⁶, —SO₂R⁶, nitro, amido, ureido, carbamato, —SR⁷, —NR⁸R⁹, or poly-alkyleneoxide.

The mono-saccharide, di-saccharide, tri-saccharide, or oligo-saccharide may be chosen from sugars that exhibit specific cancer cell targeting. Glucose and galactose have shown such selectivity.

In formula (2), X¹ is independently oxygen or sulfur, and R¹ and R² are independently selected from hydrogen and hydrocarbyl groups as defined below, except that the hydrocarbyl groups for R¹ and R² contain 1 to 4 carbon atoms. The subscript r independently represents 0 or an integer from 1 to 20.

Z is a carborane cluster. Z comprises at least two carbon atoms and at least three boron atoms, or at least one carbon atom and at least five boron atoms, within a cage structure. A carborane cluster is composed of boron and carbon atoms. Carboranes are polyhedra. Some examples of carborane clusters include the regular polyhedral carborane clusters, also known as closo structures, as well as ionized fragments of the polyhedral clusters, also known as nido structures. Some examples of the preferred carboranes of the present invention include —C₂HB₉H₁₀ or —C₂HB₁₀H₁₀, where —C₂HB₉H₁₀ is nido ortho-, meta-, or para-carborane, and —C₂HB₁₀H₁₀ is closo ortho-, meta-, or para-carborane.

Z comprises at least two carbon atoms and at least three boron atoms, or at least one carbon atom and at least five boron atoms, within a cage structure, or a substituent represented by formula (3).

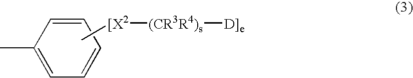

In formula (3), X² is independently oxygen or sulfur. R³ and R⁴ are independently selected from hydrogen and hydrocarbyl groups as defined below, except that the hydrocarbyl groups for R³ and R⁴ contain 1 to 4 carbon atoms. The subscript s independently represents 0 or an integer from 1 to 20, and e independently represents an integer from 1 to 4.

D is a carborane cluster. D comprises at least two carbon atoms and at least three boron atoms, or at least one carbon atom and at least five boron atoms, within a cage structure. Examples of carborane clusters are given above.

Hydrocarbyl is a straight chain or branched hydrocarbyl group containing 1 to 20 carbon atoms including, optionally, up to three double or triple bonds. Some examples of hydrocarbyl groups include methyl, ethyl, n-propyl, iso-propyl, n-butyl, iso-butyl, sec-butyl, tert-butyl, propenyl, 2-butenyl, 3-butenyl, 3-butynyl, 2-methyl-2-butenyl, n-pentyl, dodecyl, hexadecyl, octadecyl, linoleic, and eicosyl.

The hydrocarbyl group may be unsubstituted or substituted with as many hydrophilic groups as the hydrocarbyl group can tolerate, e.g. 1 to 4. Some examples of suitable hydrophilic groups include hydroxy, alkoxy, —C(O)OR⁵, —SOR⁶, —SO₂R⁶, nitro, amido, ureido, carbamato, —SR⁷, —NR⁸R⁹, and poly-alkyleneoxide. R⁵, R⁶, R⁷, R⁸, and R⁹ are independently selected from hydrogen and hydrocarbyl groups as defined above, except that the hydrocarbyl groups for R⁵, R⁶, R⁷, and R⁸ contain 1 to 4 carbon atoms.

The carbon atoms of the hydrocarbyl group may also be substituted with 1 to 4 heteroatoms. In this specification, heteroatoms are O, S, N, or NR¹⁰. The heteroatoms are generally not adjacent, and are preferably separated from each other by at least one carbon atom. Preferably, there is no more than one heteroatom for each two carbon atoms.

The non-aromatic carbocyclic or heterocyclic ring is a 4, 5, 6, 7, or 8 member carbocyclic or heterocyclic ring. The ring may be saturated, or may contain as many unsaturated (i.e., double or triple) bonds as a carbocyclic ring can tolerate.

Some examples of saturated carbocyclic rings include cyclobutane, cyclopentane, cyclohexane, and cycloheptane rings. Some examples of unsaturated carbocyclic rings include cyclobutene, cyclopentene, cyclohexene, and 1,3-cycloheptadiene rings.

The heterocyclic ring comprises as many heteroatoms, i.e. O, S, N, or NR¹⁰, as the hetercyclic ring can tolerate, e.g. 1 to 4. Some examples of saturated and unsaturated non-aromatic heterocyclic rings include pyrrolidinyl, piperidine, piperazine, tetrahydrofuran, furan, thiophene; 1,3-oxazolidine, imidazole, and pyrrole rings. The heterocyclic rings may be optionally substituted with hydrocarbyl as defined above, or with 1 to 4 hydrophilic groups, also as defined above.

The non-aromatic carbocyclic or heterocyclic rig may be a bicyclic ring. Some examples of carbocyclic rings are bicyclo[2.2.2.]octane, bicyclo[3.1.1.]heptane, bicyclo[3.3.0.]octane, and bicyclo[4.3.0.]non-3-ene. Examples of non-aromatic heterocyclic rings include 1,4 azabicyclo[2.2.2.]octane and 2-azabicyclo[3.1.1.]heptane.

An aryl group can be either aromatic carbocyclic or aromatic heterocyclic. An aromatic carbocyclic ring is preferably phenyl.

The aryl rings may be optionally substituted with hydrocarbyl as defined above to produce alkylaryl or arylalkyl groups. The aryl, alkylaryl, and arylalkyl groups may be substituted with 1 to 4 hydrophilic groups, as defined above.

Aromatic heterocyclic rings comprise 1 to 4 heteroatoms, i.e. O, S, N, or NR¹⁰. The rings are typically 5, 6, or 7 membered. Some examples of aromatic heterocyclic rings include thiophene, pyridine, oxazole, thiazole, oxazine, and pyrazine rings. The aromatic heterocyclic ring may be substituted with 1 to 4 hydrophilic groups, as defined above.

Any of the above rings may also be fused to 1 to 3 additional 5, 6, or 7 member aryl rings. Some examples of fused rings include napthalene, anthracene, phenanthrene, triphenylene, chrysene, indoline, quinoline, and tetraazanaphthalene (pteridine) rings.

In this specification, an alkoxy group contains a hydrocarbyl portion as defined above. Some examples of alkoxy groups include methoxy, ethoxy, propoxy, n-butoxy, t-butoxy, and dodecyloxy.

A polyalkylene oxide is defined according to the formula —(CH₂)_{d}—O—[(CH₂)ₑ—O—]ₓ—[(CH₂)_{f}—O—]_{y}—(CH₂)_{g}—OR′, wherein, independently, d is 0, or an integer from 1 to 10, e is 0, or an integer from 1 to 10, f is 1 to 10, g is 1 to 10, x and y are each independently 1 or 0, and R′ is either H or an hydrocarbyl group as defined previously, provided that when e is 0, then x is 0; when f is 0, then y is 0; when e is not 0, then x is 1; and when f is not 0, then y is 1. A preferable polyalkylene oxide of the invention is polyethylene oxide. Polyethylene, oxide is defined according to the formula —(CH₂)_{d}—O—[(CH₂)ₑ—O—]ₓ—[(CH₂)_{f}—O—]_{y}—(CH₂)_{g}—OR′, wherein, independently, d is 0 or 2, e is 0 or 2, f is 0 or 2, g is 2, x and y are each independently 1 or 0, and R′ is either H or an ethyl group, provided that when e is 0, then x is 0; when f is 0, then y is 0; when e is not 0, then x is 1; and when f is not 0, then y is 1.

In formula (1), M may be two hydrogen ions, a single monovalent metal ion, or two monovalent metal ions. Some examples of suitable monovalent metal ions include Li⁺¹, Na⁺¹, K⁺¹, Cu⁺¹, Ag⁺¹, Au⁺¹, and Tl⁺¹. When M is a single monovalent metal ion, the resulting porphyrin-metal complex anion is charge-balanced by a counter cation. Some examples of counter cations include any of the foregoing monovalent metal ions, and ammonium and phosphonium cations, such as tetramethylammonium, tetrabutylammonium, tetraphenylammonium, tetramethylphosphonium, tetrabutylphosphonium, and tetraphenylphosphonium. The counter cation may be either bound or associated in some form with the porphyrin-metal complex.

M may also be a divalent metal ion. Some examples of suitable divalent metal ions include V⁺², Mn⁺², Fe⁺², Ru⁺², Co⁺², Ni⁺², Cu⁺², Pd⁺², Pt⁺², Zn⁺², Ca⁺², Mg⁺², Sr⁺², and Ba⁺².

Alternatively, M may be a trivalent, tetravalent, pentavalent, or hexavalent metal ion. Some examples of suitable trivalent metal ions include Gd⁺³, Y⁺³, In⁺³, Cr⁺³, Ga⁺³, Al⁺³, Eu⁺³, and Dy⁺³. Some examples of suitable tetravalent metal ions include Tc⁺⁴, Ge⁺⁴, Sn⁺⁴, and Pt⁺⁴. An example of a suitable pentavalent metal ion is Tc⁺⁵. Some examples of suitable hexavalent metal ions include W⁺⁶, Tc⁺⁶, and Mo⁺⁶.

The resulting porphyrin-metal complex cation is charge-balanced by an appropriate number of counter anions. The anions may be monoanions, dianions, or trianions. For example, a porphyrin-metal complex cation derived from a trivalent metal ion may be charge-balanced by a single counter monoanion, and such a complex derived from a tetravalent metal ion may, for example, be charge-balanced by a single counter dianion or two counter monoanions, and so on.

Some examples of suitable counter monoanions include chloride, perchlorate, sulfate, nitrate, and tetrafluoroborate. Some examples of suitable counter dianions include oxide, sulfide, or a porphyrin compound containing a divalent negative charge. The porphyrin compound containing a divalent negative charge may be a porphyrin compound of the present invention with the proviso that M is absent. An example of a suitable counter trianion includes phosphate.

The counter monoanion, dianion, or trianion may be either bound or associated in some form with a carborane-containing porphyrin compound of the present invention. The carborane-containing porphyrin compound may also be bound to or associated with neutrally charged molecules, such as molecules of solvation, for example, water, acetonitrile, methanol, and so on.

M may be a radioactive metal ion imageable by single photon emission computed tomography (SPECT) or positron emission tomography (PET). Some examples of radioactive metals suitable for SPECT are ⁶⁷Cu, ^{99m}Tc, ¹¹¹In, and those for PET include ⁶⁴Cu, ⁵⁵Co. M may also be a radioactive metal useful as a radiopharmaceutical for therapy. Some examples of radioactive metals suitable for such therapy include ⁹⁰Y, ¹⁸⁸Re, ⁶⁷Cu.

M may also be a paramagnetic metal ion detectable by magnetic resonance imaging (MRI). Some examples of such metals include Mn, Fe, Co, and Gd.

In addition, M may be a metal ion suitable for boron neutron capture therapy (BNCT) or photodynamic therapy (PDT); or a combination thereof. The metal ions suitable for BNCT include those described thus far, with the exclusion of those that are photoactive, such as Zn and Sn. Such photoactive metals, and particularly those with long-lived triplet states, are preferable for PDT. Since the dosage for BNCT is 100 to 1000 times greater than the dosage for PDT, a significant accumulation of photoactive metal in the skin could result if such photoactive metals were used in BNCT. Such an accumulation of photoactive metal may cause biological damage.

The invention also relates to methods of treating tumors. In a preferred embodiment, the method of treating malignant tumors, especially brain tumors, is BNCT. Clinical BNCT for malignant brain tumors was carried out at the Brookhaven National Laboratory Medical Department using p-boronophenylalanine (BPA) as the boron carrier (Chanana et al., Neurosurgery, 44, 1182-1192, 1999).

The description of BNCT from the Chanana et al. article is incorporated herein by reference. Those having ordinary skill in the art can readily adapt the method to the compounds of the invention.

In BNCT of malignant brain tumors following the method of the present invention, for example, the patient is first given an infusion of a carborane-containing porphyrin of formula (1), which is highly enriched in boron-10. The carborane-containing porphyrin is then concentrated preferentially in the brain tumor within the effective irradiation volume, which, for brain tumors may be a substantial part of the brain. For example, tumors located in most or all of one hemisphere and some or all of the contralateral hemisphere of the brain can accumulate boronated porphyrins.

The tumor area is then irradiated with thermalized neutrons (primary irradiation), some of which are captured by the boron-10 concentrated in the tumor. The relative probability that the slow-moving thermal neutrons will be captured by the boron-10 nuclide is high compared to the probability of capture by all of the other nuclides normally present in mammalian tissues, provided that boron-10 concentrations in tumor tissues is greater than 30 μg/g.

Since a minuscule proportion of the boron-10 nuclei in and around a tumor undergoes the nuclear reaction immediately after capturing a neutron, a high concentration of boron-10 in the targeted tissue is necessary for BNCT to be clinically effective. Therefore, to maximize the concentration of boron-10 in the targeted tissue, the carborane clusters are highly enriched in boron-10. Specifically, the boron in the carborane cluster is enriched to at least 95 atom % in boron-10.

The porphyrin compounds of the present invention that have been tested in vivo are non-toxic at theoretically therapeutic effective doses. The higher selectivity and lower toxicity of the carborane-containing porphyrins of the present invention allow for the selective destruction of tumor tissue with minimal disruption of normal tissues and tissue function when irradiated.

Another advantage of the carborane-containing porphyrins of the present invention is their increased polarity, imparted through polar saccharide groups on the phenyl rings. The greater polarity of such groups render the tetraphenyl porphyrin compounds less lipophilic, which effects a reduction of the amount of an emulsifying co-solvent during administration. Therefore, the microlocalization within the tumor cell may be improved yielding a higher relative biological effect.

Additionally, the saccharide groups on the porphyrin rings are believed to alter cellular membrane receptor targeting which may allow the porphyrin complex to attach to receptors on the cellular membranes of cancerous cells. No toxicities were seen in biodistribution/toxicological screens in a murine tumor model. Significant boron concentrations were present in the tumor and very little in the blood and brain.

In addition, when X¹ or X² of the porphyrins are oxygen, the ether linkages in the carborane-containing porphyrins of the present invention are more polar than carbon-carbon linkages and therefore, provide a further reduction in lipophilicity. At the same time, the ether linkages possess nearly the same resistance to hydrolysis and other forms of chemical attack as a carbon-carbon linkage.

To accumulate the requisite amount of a compound of the present invention in a tumor, generally a systemically injected or infused dose of about 10-50 milligrams of boron-10 per kg body weight in a pharmaceutically acceptable carrier is administered to a patient. The carrier may include such commercially available solvents as Cremophor EL, propylene glycol, Tween 80, polyethylene glycol, or liposomes. The compound is administered in one or more doses, the last dose being given between about one hour to one week prior to the epithermal neutron irradiation.

The timing of the neutron exposure depends upon the concentration of the porphyrin in the blood, which decreases more rapidly with time than the porphyrin concentration in the tumor. However, the timing of the administration of the carborane-containing porphyrin depends on various considerations that are well known to those skilled in the art of clinical BNCT, including the pharmacokinetic behavior of the compound, (e.g., the rate of absorption of the compound into the tumor and into the tumor vasculature) and the rate of excretion from, and/or metabolism of the compound in the tumor and various other tissues that absorb the compound.

In another preferred embodiment, the method of treating malignant tumors of the present invention is PDT. PDT is a bimodal cancer treatment based on the selective accumulation of a porphyrin in a tumor, followed by irradiation of the tumor with laser red light. Upon activation with light, an electron of the porphyrin is excited from the singlet ground state to a singlet excited state. The electron then can either return to the singlet ground state with the emission of light causing fluorescence, or it can change its spin via intersystem crossing to the triplet state. In the decay of the triplet back down to the ground state singlet, it can transfer energy to ground state triplet dioxygen which forms the highly reactive singlet oxygen. Biomolecules that react most readily with singlet oxygen include unsaturated lipids and alpha amino-acid residues, both of which are major constituents of biological membranes. Beyond a certain reversible or repairable threshold, damage to membranes, especially to endothelial cell membranes, can lead to local vascular thrombosis and shutdown of blood circulation.

In using PDT in the present invention, the patient is first given an injection or infusion of a photosensitizing carborane-containing porphyrin of formula (1). Fiber-optic probes are then used to illuminate the tumor tissue. For malignant tumors,it is preferable that the PDT photosensitizers have optical absorbance peaks at sufficiently long wavelengths for maximum penetration to the depth of the tumor.

In a preferred embodiment, the therapeutic treatment of malignant tumors is augmented by the use of SPECT or PET. In SPECT, the patient is first given an infusion or injection of a compound of formula (1) wherein M is a gamma-emitting radioactive metal ion. The patient's head is then scanned noninvasively and the radionuclide concentration, and hence indirectly, the average boron concentration, in each pixel or voxel representing brain or brain tumor tissue is imaged. Contour lines representing zones of equal boron-10 concentration can thereby be drawn on each image of the brain.

SPECT of the brain is at least one order of magnitude more sensitive to isotopic tracers than is conventional radiography or computerized tomography. In addition, SPECT results, as opposed to results from conventional radiography, can be analyzed to provide quantitative information either in defined volumes or voxels of the brain images, in the concentrations of boron relevant to BNCT treatment planning and implementation. SPECT scanning can indicate the presence of a tumor in the patient, as well as its location in the brain or elsewhere in the body. SPECT scanning is noninvasive, fast, and convenient.

However, the positron emitting PET-imageable radioisotope Cu-64, is more readily available than is Cu-67, used in SPECT. Because of the much greater availability of Cu-64, we have carried out preclinical PET studies using a Cu-64 labeled porphyrin.

In another preferred embodiment, the therapeutic treatment of malignant tumors is augmented by the use of MRI. In MRI, a patient is first given an infusion or injection of a solution containing a carborane-containing porphyrin of formula (1) wherein M is a suitable paramagnetic metal ion. For a brain tumor, the patient's head is then scanned and the paramagnetic metal ion concentration, and thus, boron concentration in the brain is imaged and quantified. MRI utilizing the compounds of the present invention may permit rapid enhanced targeting and treatment planning for neutron irradiation in BNCT before, during and after infusion when the boronated compound is being redistributed in blood, tumor, and healthy tissue.

The carborane-containing porphyrins of the present invention are synthesized through a series of separate steps. Provided below is first, a summary of the synthetic steps required for the preparation of the preferred carborane-containing porphyrins of the present invention, wherein two of Y¹, Y², Y³, and Y⁴ are represented by the formula

—X—(CR¹R²)ᵣ-Z (2)

and the two not represented by formula (2) are glucose or galactose. The synthetic summary provides general methods for synthesizing compounds of the invention, and thereby includes several different specific ways to achieve any one synthesis. For example, different starting materials may be used to synthesize the same product. Each starting material may require a different set of reaction conditions such as temperature, reaction time, solvents, and extraction and purification procedures. Those skilled in the art will readily be able to ascertain such reaction conditions.

The specific examples describe a preferred method for synthesizing the compounds of the present invention. The scope of this invention is not to be in any way limited by the examples set forth herein. For example, glycosylated carborane-containing tetraphenylporphyrin compounds can be synthesized by using a mixture of different benzaldehyde or dibenzaldehyde starting materials and proceeding with a similar synthetic reaction as shown in reaction scheme 8. Different saccharide starting materials can also be used in Reaction Scheme 11 to create various saccharide substituents.

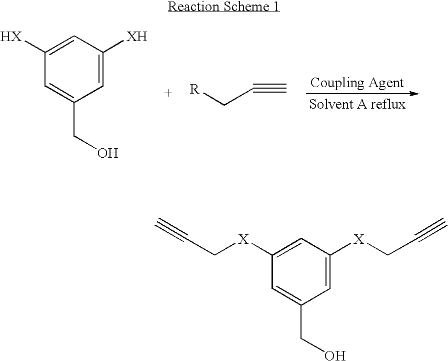

where X is either O or S, solvent A is preferably a polar aprotic solvent such as acetone, and R is a halogen, preferably Cl. The coupling agent is any compound, mixture, or sequence of compounds capable of coupling a phenol or thiophenol and an alkyl halide to produce an ether. Some coupling agents may not require reflux conditions or a polar aprotic solvent. Preferably, the coupling agent is a mixture of potassium carbonate and potassium iodide (K₂CO₃/KI).

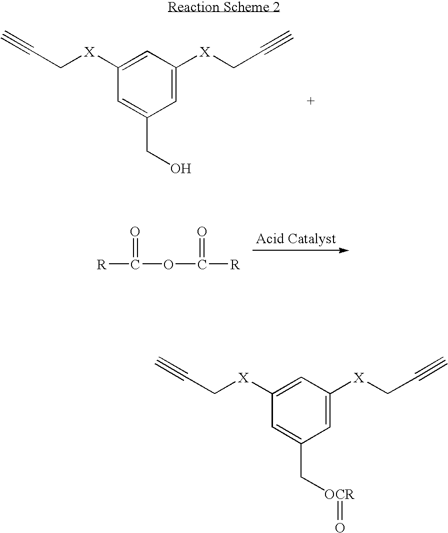

where X is as defined above. The R groups on the anhydride may be the same or different, and selected from hydrocarbyl, non-aromatic carbocyclic, non-aromatic heterocyclic, or aryl. A preferred anhydride is acetic anhydride wherein R is methyl. The acid catalyst may be any Bronsted-Lowry (proton donating) acid that does not interfere with conversion of the alcohol to the ester product. Preferably, the acid catalyst is sulfuric acid, H₂SO₄.

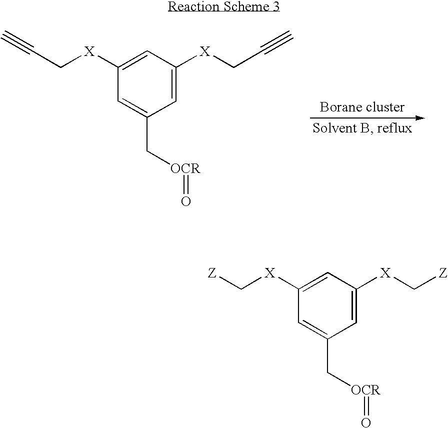

where X is as defined above. Solvent B is preferably a polar, aprotic solvent, preferably acetonitrile, and R is as defined in reaction scheme 2. The borane cluster (Z) is any cluster comprising at least three boron atoms within a cage structure. For example, the borane cluster can be decaborane, B₁₀H₁₄. The borane cluster reacts with the triple bond of the propargyl starting material to form the carboranyl product. Thus, in the case of decaborane, Z represents the carborane —C₂HB₁₀H₁₀. Z represents any carborane cluster comprising at least two carbon atoms and at least three boron atoms, or at least one carbon atom and at least five boron atoms, within a cage structure. For example, the carborane cluster may be —C₂HB₉H₁₀ or —C₂HB₁₀H₁₀, wherein —C₂HB₉H₁₀ is nido ortho-, meta-, or para-carborane, and —C₂HB₁₀H₁₀ is closo ortho-, meta-, or para-carborane.

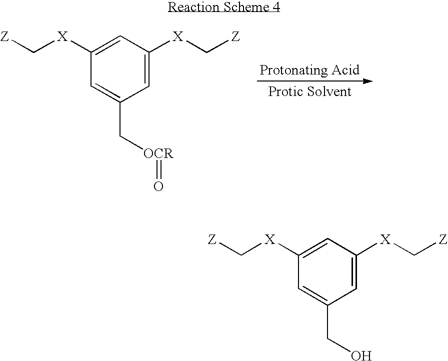

where X and Z are as defined above, and R is as defined in reaction scheme 2. The protonating acid is any acid, acid mixture, or sequence of acid additions capable of converting the ester into the alcohol product. Preferably, the protonating acid is concentrated HCl. The protic solvent may be, for example, an alcohol such as methanol.

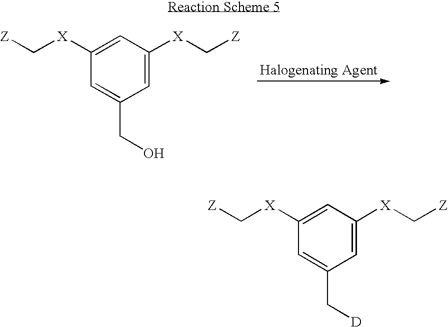

where X and Z are as defined above, and D is a halogen. The halogenating agent is any agent capable of converting the hydroxy substituent of the starting material to a halogen. Preferably, the halogenating agent is a 1:1 mixture of carbon tetrabromide and triphenylphosphine, wherein D becomes a bromide. The reaction is performed preferably in an ether solvent, such as tetrahydrofuran (THF).

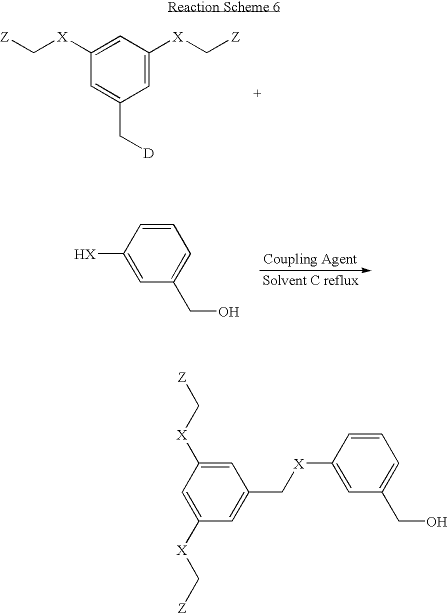

where X, Z, and D are as defined previously. Solvent C is preferably a polar, aprotic solvent such as acetone. The coupling agent is any compound, mixture, or sequence of compounds capable of coupling a phenol or thiophenol and an alkyl halide to produce an ether. Some coupling agents may not require reflux conditions or a polar, aprotic solvent. Preferably, the coupling agent is a mixture of potassium carbonate and potassium iodide (K₂CO₃/KI).

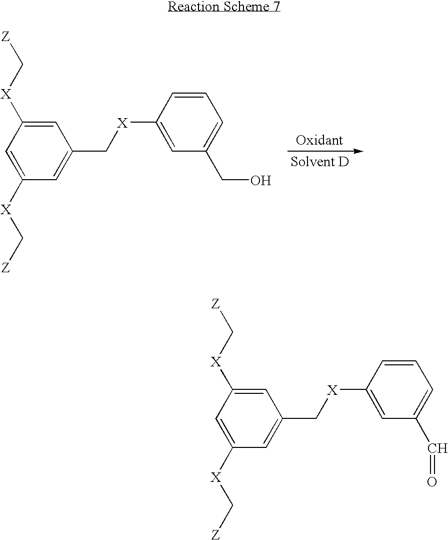

where X and Z are as previously defined. The oxidant is any oxidizing compound capable of selectively converting a primary alcohol to an aldehyde, preferably 2,3-dichloro-5,6-dicyano-1,4-benzoquinone (DDQ) or pyridinium chlorochromate (PCC). Solvent D is a non-polar solvent, preferably dichloromethane (DCM).

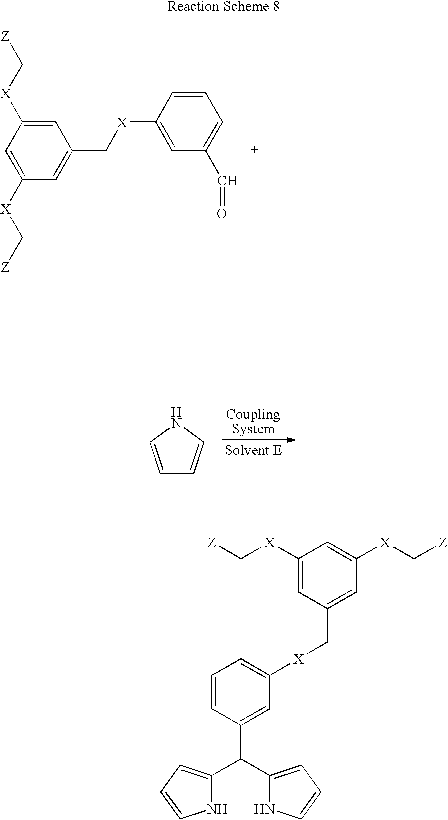

wherein X and Z have been previously defined. The coupling system preferably comprises a Lewis acid (electron acceptor) such as boron trifluoride (BF₃) or trifluoroacetic acid (TFA) to form the intermediate porphyrinogen from the pyrrole. Solvent E is a nonpolar solvent, preferably DCM.

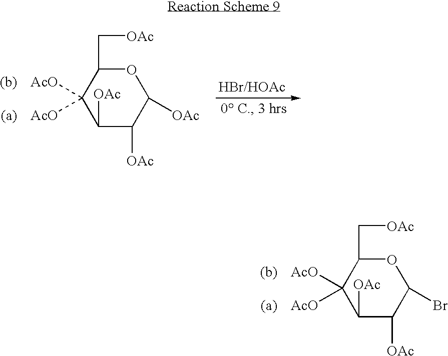

where (a) represents glucose and (b) represents galactose.

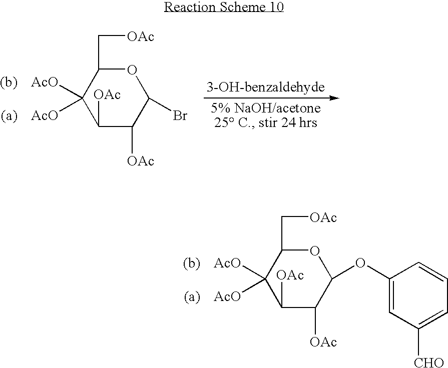

where (a) and (b) are as previously defined.

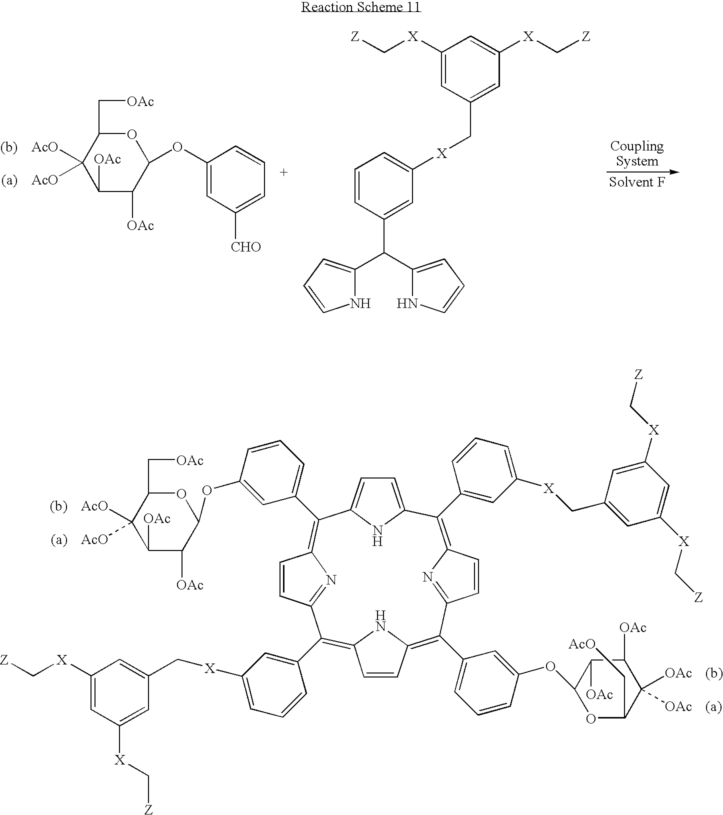

wherein X, Z, (a), and (b) are as previously defined. The coupling system preferably comprises a Lewis acid (electron acceptor) such as boron trifluoride (BF₃) or trifluoroacetic acid (TFA) to form the intermediate porphyrinogen from the pyrrole, and benzaldehyde and an oxidizing agent such as 2,3-dichloro-5,6-dicyano-1,4-benzoquinone (DDQ) to oxidize the porphyrinogen to porphyrin. Solvent F is a nonpolar solvent, preferably DCM.

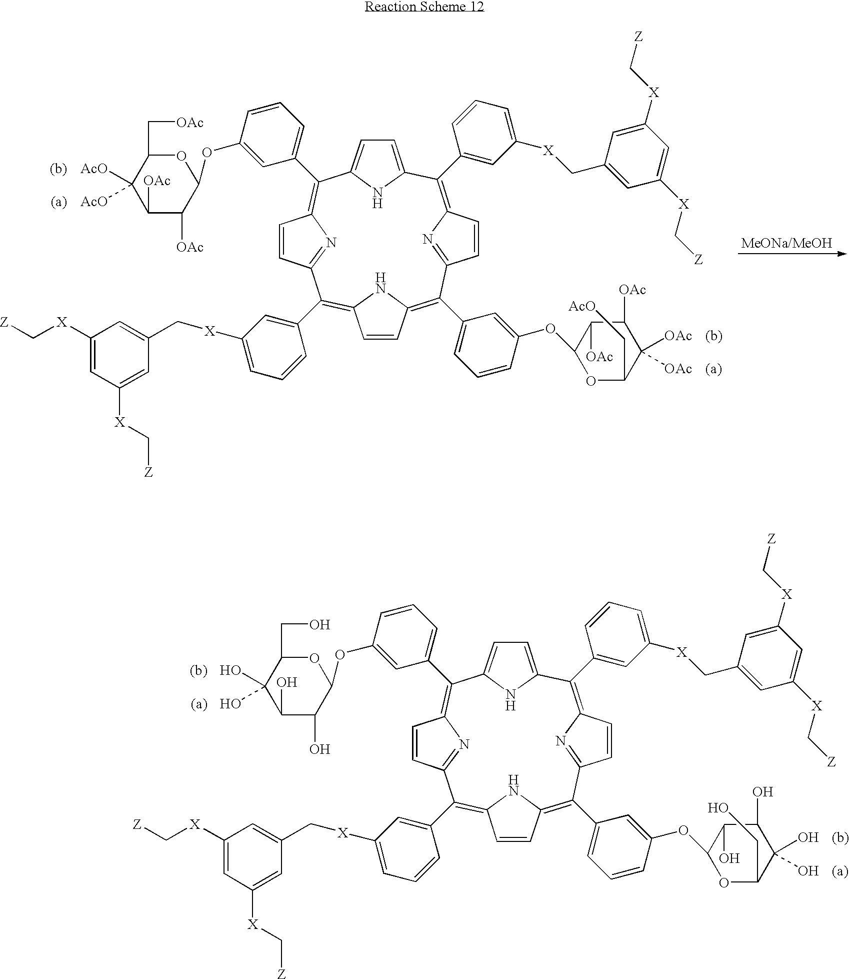

where X, Z, (a), and (b) are as previously defined.

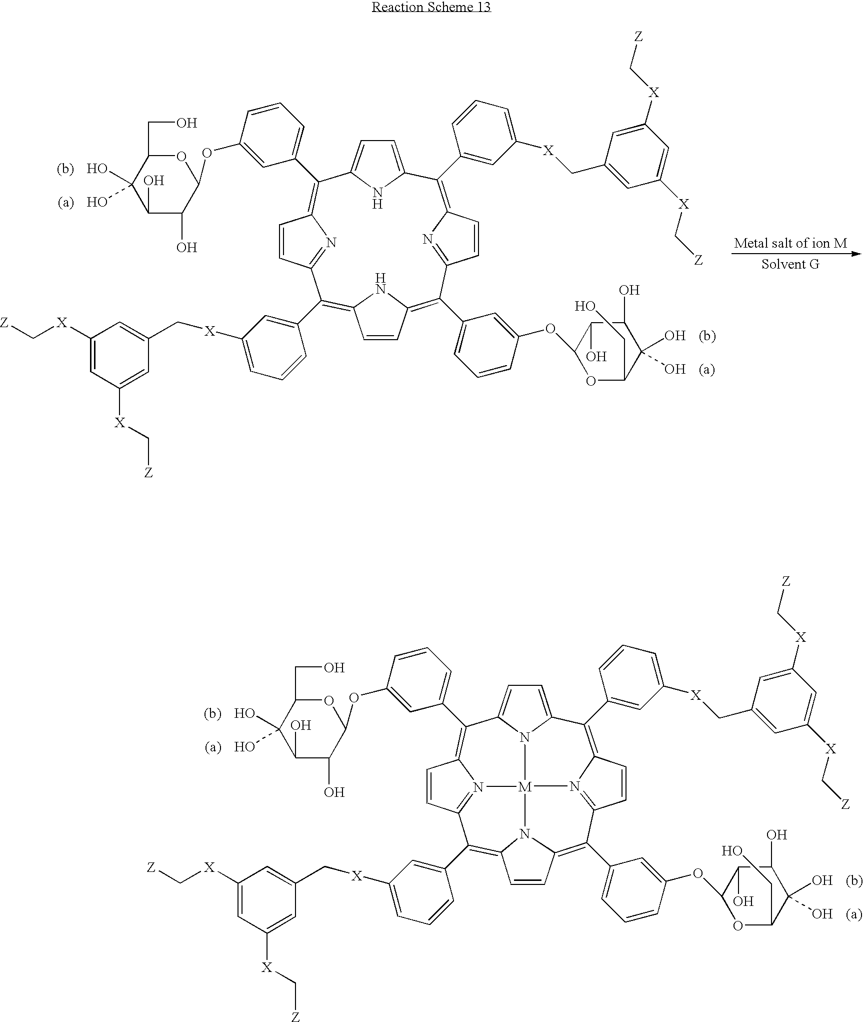

wherein X, Z, (a), and (b) are as previously defined. In a preferred embodiment, M is selected from the group consisting of vanadium (V), manganese (Mn), iron (Fe), ruthenium (Ru), technetium (Tc), chromium (Cr), platinum (Pt), cobalt (Co), nickel (Ni), copper (Cu), zinc (Zn), germanium (Ge), indium (In), tin (Sn), yttrium (Y), gold (Au), barium (Ba), tungsten (W), and gadolinium (Gd). In a more preferred embodiment, M is copper (Cu) or nickel (Ni). The metal salt used contains the metal ion M to be chelated to the porphyrin. For example; for the compound where M is desired to be copper, copper acetate, i.e., Cu(OAc)₂.H₂O, may be used as the metal salt. Solvent G is any solvent or solvent mixture capable of at least partially solubilizing the porphyrin and metal salt, and that does not interfere with incorporating the metal into the porphyrin.

For example, using reaction schemes 1-13, and as will be seen through examples 1-13, the 4-carborane-containing porphyrin, i.e., porphyrin (XXIII), has been prepared. Porphyrin XXIII has the following structure:

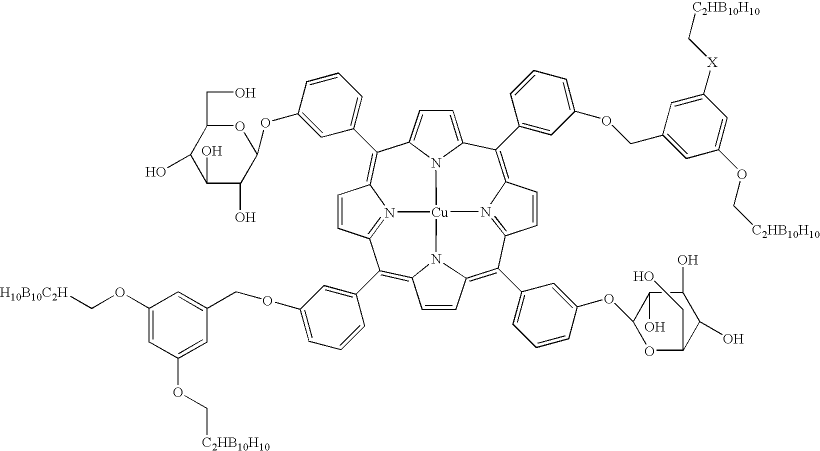

Porphyrin XXIII shown above. In this case, a, b, c, and d are 1; Y¹ and Y³ are represented by formula (2); Z is represented by formula (3); X¹ and X² are O; r and s are 1; R¹, R², R³, and R⁴ are H; D is the —C₂HB₁₀H₁₀ carborane; e is 2; Y¹, Y², Y³, and Y⁴ are on the meta positions of the phenyl ring; Y² and Y⁴ are β-D-glucose; and M is Cu.

### EXAMPLES

Examples have been set forth below for the purpose of illustration and to describe the best mode of the invention at the present time. The scope of the invention is not to be in any way limited by the examples set forth herein.

### A. Synthesis of copper (II) 5,15-bis[3-(o-carboranylmethoxy)phenyl]-10,20-bis[3-(β-D-glucosyl)phenyl]porphyrin (VII)

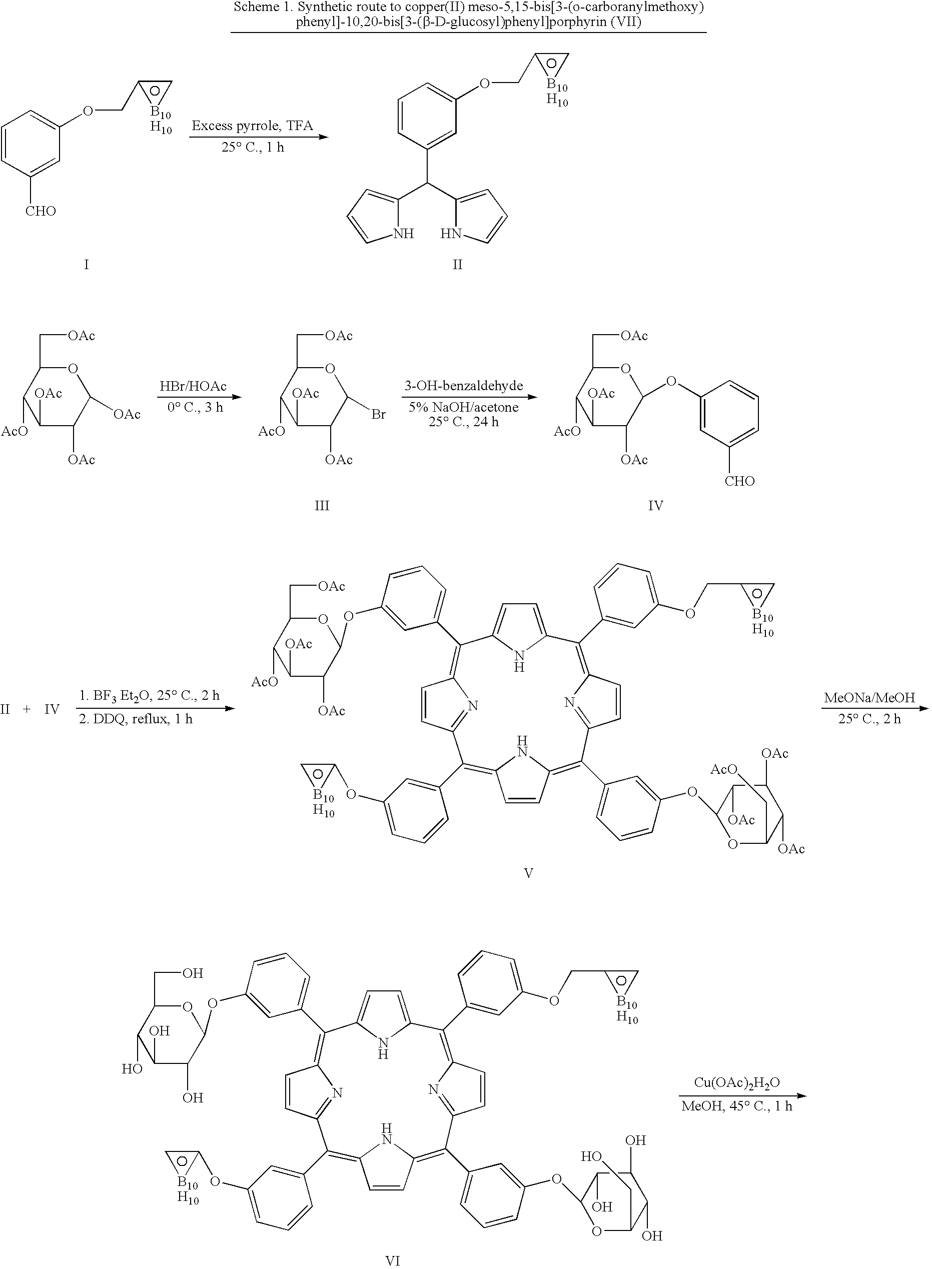

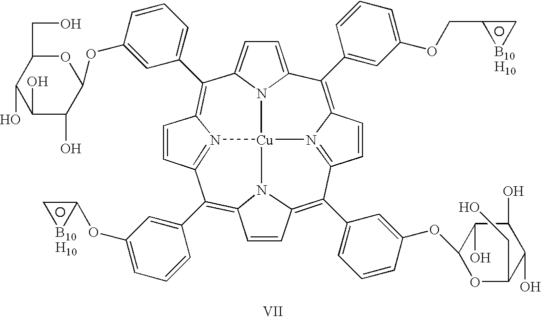

### Example 1

### Synthesis of 3-o-carboranyloxymethylbenzaldehyde (I)

3-o-carboranyloxymethylbenzaldehyde (I) was synthesized as previously described [M. Miura et al, Tet Let, 1990, 31, 2247-2250]

### Example 2

### Synthesis of 3-(o-carboranylmethoxy)phenyl dipyrromethane (II)

A solution of freshly distilled pyrrole (17 mL, 250 mmol) and 3-o-carboranyloxymethylbenzaldehyde (I) (1.38 g, 50 mmol) in dry dichloromethane [DCM] (100 mL) was bubbled with argon for 20 min, trifluoroacetic acid [TFA] (330 μL, 3 mmol) was added and the mixture was stirred for 1 h. Solvents were then evaporated under vacuum; the residue was dissolved in DCM, and worked up resulting in a yellow oil, which solidified upon standing. This was further purified using a silica pad eluting with hexane/DCM (2/1, v/v). The first fraction was collected yielding 1.9 g, 97%.

The product gave the following ¹H NMR spectrum in ppm (in CDCl₃, solvent): 1.4-3.4 (m, 10H, carborane H), 4.03 (d, 1H, —CCHB₁₀H₁₀), 4.34 (s, 2H, OCH₂), 5.42 (s, 1H, CH), 5.89 (m, 1H, β-pyr-H), 6.16 (m, 1H, β-pyr-H), 6.70 (m, 2H, α-pyr-H), 6.70 (m, 2H, ArH), 6.73 (m, 1H, ArH), 6.89 (m, 1H, ArH), 7.24 (ArH), 7.90 (NH). The product gave the following proton-decoupled ¹³C NMR spectrum in ppm (in CDCl₃, solvent): 44.1(CH), 58.0(CH₂), 69.2(-CCHB₁₀H₁₀), 71.6(-CCHB₁₀H₁₀), 107.6 (pyr), 108.8 (pyr), 113.2 (ArC), 115.1 (ArC), 117.7 (pyr), 122.8 (ArC), 130.2 (ArC), 132.1(pyr), 144.6 (ArC), 15.7.5 (ArC). C₁₈H₂₈N₂OB₁₀ requires 394.5, MS (FAB) m/e 394.7 (M⁺).

### Example 3

### Synthesis of α-D-Glucose tetraacetate bromide (III)

Hydrogen bromide in acetic acid (30%, 14 mL) was added to α-D pentaacetyl glucose (3.9 g, 0.010 mol) at 0° C. and the mixture stirred at room temperature for 3 h. The solution was concentrated under vacuum at 40° C. and then 60° C. The crude oil was dissolved in DCM and quickly washed with cold water, and worked up. The oil solidified upon standing yielding a white solid, 4.1 g, 100%. The bromide was stored in freezer and used without further purification.

The product gave the following ¹H NMR spectrum in ppm (in CDCl₃, solvent): 2.04 (s, 3H, CH₃), 2.06 (s, 3H, CH₃), 2.10 (d, 6H, CH₃), 4.13(d-d, 1H, CH), 4.31 (d-d, 1H, CH), 4.83(d-d, 1H, CH), 5.16 (t, 1H, CH), 6.61 (m, 1H, CH). The product gave the following proton-decoupled ¹³C NMR spectrum in ppm (in CDCl₃, solvent): 20.7 CH₃), 20.8 CH₃), 20.8 CH₃), 20.8 (CH₃), 61.2(CH₂), 67.4(CH), 70.4(CH), 70.8(CH), 72.3(CH), 86.8(CH), 169.6 (CO), 169.9 (CO), 170.0 (CO), 170.7 (CO). C₁₄H₁₉BrO₉ requires 411.2, MS (FAB) m/e 411.4 (M⁺).

### Example 4

### Synthesis of 3-(β-D-glucosyl tetraacetate)benzaldehyde (IV)

α-D-glucose tetraacetate bromide (III) (4.1 g, 10 mmol) and 3-hydroxybenzaldehyde (2.4 g, 20 mmol) were dissolved in acetone (10 mL), under a nitrogen atmosphere while stirring at room temperature. A 5% NaOH solution (16 mL, 20 mmol) was added dropwise over 15 min and the solution was allowed to stir overnight. TLC showed starting aldehyde and a new product. The mixture was poured into ice, the product was extracted with DCM and washed with 5% NaOH, water, dried over anhydrous sodium sulfate, and the solvents were removed in vacuo resulting in a colorless oil, which solidified upon cooling yielding 2.3 g, 52%. It was recrystallized in methanol.

The product gave the following ¹H NMR spectrum in ppm (in CDCl₃, solvent): 2.04 (s, 3H, CH₃), 2.06 (m, 6H, CH₃), 2.10 (s, 3H, CH₃), 3.94 (m, 1H, CH), 4.24 (m, 2H, CH₂), 5.17 (m, 2H, CH), 5.31 (m, 2H, CH), 7.27 (s, 1H, ArH), 7.51(m, 2H, ArH), 7.58 (s, 1H, ArH), 10.0 (s, 1H, ArCHO). The product gave the following proton-decoupled ¹³C NMR spectrum in ppm (in CDCl₃, solvent): 20.8 (CH₃), 62.2 (CH₂), 68.4 (CH), 71.3 (CH), 72.5 (CH), 72.8 (CH), 98.8 (CH), 115.4 (ArC), 123.9 (ArC), 126.2 (ArC), 130.5 (ArC), 138.2 (ArC), 157.5 (ArC), 169.5(CO), 169.6 (CO), 170.4 (CO), 170.9 (CO), 191.6 (CO). C₂H₂₄O₁₁ requires 452.4, MS (FAB) m/e 452.5 (M⁺).

### Example 5

### Synthesis of meso-5,15-bis[3-(o-carboranylmethoxy)phenyl]-10,20-bis[3-(β-D-glucose tetraacetate)phenyl]porphyrin (V)

3-(β-D-Glucosyl tetraacetate)benzaldehyde (IV) (450 mg, 1 mmol), 3-(o-carboranylmethoxy)phenyl dipyrromethane (II) (397 mg, 1 mmol) in DCM (100 mL) in a dry 300 mL round bottom flask was deoxygenated by bubbling argon into the vigorously stirring solution for 15-20 min. BF₃.Et₂O (36 μL, 0.3 mmol) was added and the solution was allowed to stir for 2 hours. Dichloro-dicyano-benzoquinone [DDQ] (227 mg, 1 mmol) was added (solution turned dark immediately) and the solution was refluxed for one hour. TLC indicated a major red product. The reaction mixture solution was cooled and then eluted through a pad of ˜30 mL silica using DCM as eluent, and the red band is finally washed off with excess DCM. The solvents were removed in vacuo. Another silica pad may be used to ensure the purity. The yield was 90 mg, 11%.

The product gave the following ¹H NMR spectrum in ppm (in CDCl₃, solvent): −0.86 (s, 2H, NH), 1.4-3.4 (m, 20H, BH), 1.99-2.08 (t, 24H, CH₃), 3.80-4.14 (m, 2H, sugar, CH), 4.10 (s, 2H, —CCHB₁₀H₁₀), 4.15 (m, 2H, sugar CH), 4.60 (6H, sugar CH₂ and B₁₀H₁₀CH₂), 5.20-5.37(m, 2H, sugar, CH), 5.37 (4H, sugar, OCH₂), 7.27 (2H, ArH), 7.70 (8H, ArH), 7.90 (4H, ArH), 8.85-8.91 (8H, pyr-H). The product gave the following proton-decoupled ¹³C NMR spectrum in ppm (in CDCl₃, solvent): 20.7 (sugar, CH₃), 20.8 (sugar, CH₃), 20.9 (sugar, CH₃), 21.0 (sugar, CH₃), 58.1 (B₁₀H₁₀CH₂), 62.1 (sugar CH₂), 68.4 (sugar CH), 69.6 (CCHB_{10l H}₁₀), 71.5 (sugar CH), 71.6(-CCHB₁₀H₁₀), 72.4 (sugar CH), 72.9 (sugar CH), 99.4 (sugar CH), 114.5 (ArC), 116.8 (ArC), 119.5 (ArC), 123.3 (ArC), 119.8 (ArC), 121.3 (ArC), 128.0 (ArC), 128.2 (ArC), 129.3 (ArC), 130.1 (ArC), 143.6 (pyr), 143.9 (pyr), 155.6 (ArC), 155.7(ArC), 169.6 (CO), 170.3 (CO), 170.4 (CO), 170.6 (CO). The ultraviolet-visible absorbance spectrum of the product showed the following peaks in nanometers of wavelength (DCM): 417.9, 514, 548, 589, 644. C₇₈H₉₀B₂₀N₄O₀₂₂ requires 1651.8, MS (FAB) m/e 1651.2 (M⁺).

### Example 6

### Synthesis of meso-5,15-Bis[3-(o-carboranylmethoxy)phenyl]-10,20-bis[3-(β-D-glucosyl)phenyl]porphyrin (VI)

Meso-5,15-bis[3-(o-carboranylmethoxy)phenyl]-10,20-bis[3-(β-D-glucose tetraacetate)phenyl]porphyrin (V) (80 mg, 0.0483 mmol) was dissolved in methanol (20 mL) and a catalytic amount of 0.22 M MeONa/MeOH solution (160 μL) was added. After the solution stirred at room temperature for 2 hours, 2 drops of water were added to quench the solution. The solvents were removed by rotary evaporation (T<60° C.). TLC showed a single product with only a trace of baseline contamination (eluent:acetone/ethyl acetate: 1/1). The residue is purified by a silica pad (eluent: hexane/acetone/ethyl acetate: 1/1/1), yielding a red solid, 53 mg, 83%.

The product gave the following ¹H NMR spectrum in ppm (DMSO, 2.50 ppm): −2.89(s, 2H, NH), 1.4-3.4 (m, 40H, carborane H), 3.03 (m, 1H, sugar H), 3.30 (m, 1H, sugar CH), 3.41 (s, 11H, 10 sugar OH and 1 sugar CH), 3.56 (m, 5H, 4 sugar CH₂ and 1 sugar CH), 3.76 (m, 1H, sugar CH), 4.57 (m, 1H, sugar CH), 4.60 (—CCHB₁₀H₁₀), 4.85 (s, 8H, B₁₀H₁₀CH₂), 5.06 (m, 1H, sugar CH), 5.17 (m, 1H, sugar CH), 5.25 (m, 1H, sugar CH), 5.39 (s, 4H, ArCH₂), 5.49 (m, 1H, sugar CH), 7.50-7.59 (m, 4H, ArH), 7.76 (m, 4H, ArH), 7.91 (m, 8H, ArH), 8.91 (s, 8H, pyH). The product gave the following proton-decoupled ¹³C NMR spectrum in ppm (in DMSO, solvent): 55.8 (sugar CH₂), 60.8 (sugar CH), 61.9 (B₁₀H₁₀CH₂), 69.6 (CCHB₁₀H₁₀), 69.8 (sugar CH), 71.6(-CCHB₁₀H₁₀), 76.7 (sugar CH), 77.0 (sugar CH), 100 (sugar CH), 112.9 (ArC), 115.0 (ArC), 117.1 (ArC), 119.5 (ArC), 119.8 (ArC), 120.9 (ArC), 122.5 (ArC), 128.1 (ArC), 128.3 (ArC), 131.4 (ArC), 142.4 (pyr), 142.7 (pyr), 155.7 (ArC), 156.0 (ArC). 208.4 (DMSO). The ultraviolet-visible absorbance spectrum of the product showed the following peaks in nanometers of wavelength (in acetone solvent): 415, 511, 546, 588, 643.

### Example 7

### Synthesis of copper (II) meso-5,15-bis[3-(o-carboranylmethoxy)phenyl]-10,20-bis[3-(β-D-glucosyl)phenyl]porphyrin (VII)

To a solution of meso-5,15-bis[3-(o-carboranylmethoxy)phenyl]-10,20-bis[3-(β-D-glucosyl)phenyl]porphyrin (VI) (53 mg, 0.040 mmol) dissolved in methanol, (20 mL) copper acetate monohydrate (10 mg, 0.050 mmol) was added, and the color changed from purple to red immediately. The mixture was warmed for 1 hour and monitored by UV-vis spectroscopy. The reaction was worked up, solvents were removed and the residue was dissolved in hexane/acetone/ethyl acetate (1/1/1) and eluted through a silica pad using the same solvent mixture yielding a red solid, 40 mg, 73%.

The ultraviolet-visible absorbance spectrum of the product showed the following peaks in nanometers of wavelength (in acetone solvent): 412, 537.

### B. Synthesis of Copper (II) meso-5,15-bis[3-(o-carboranylmethoxy)phenyl]-10,20-bis[3-(β-D-galactosyl)phenyl]porphyrin (XII)

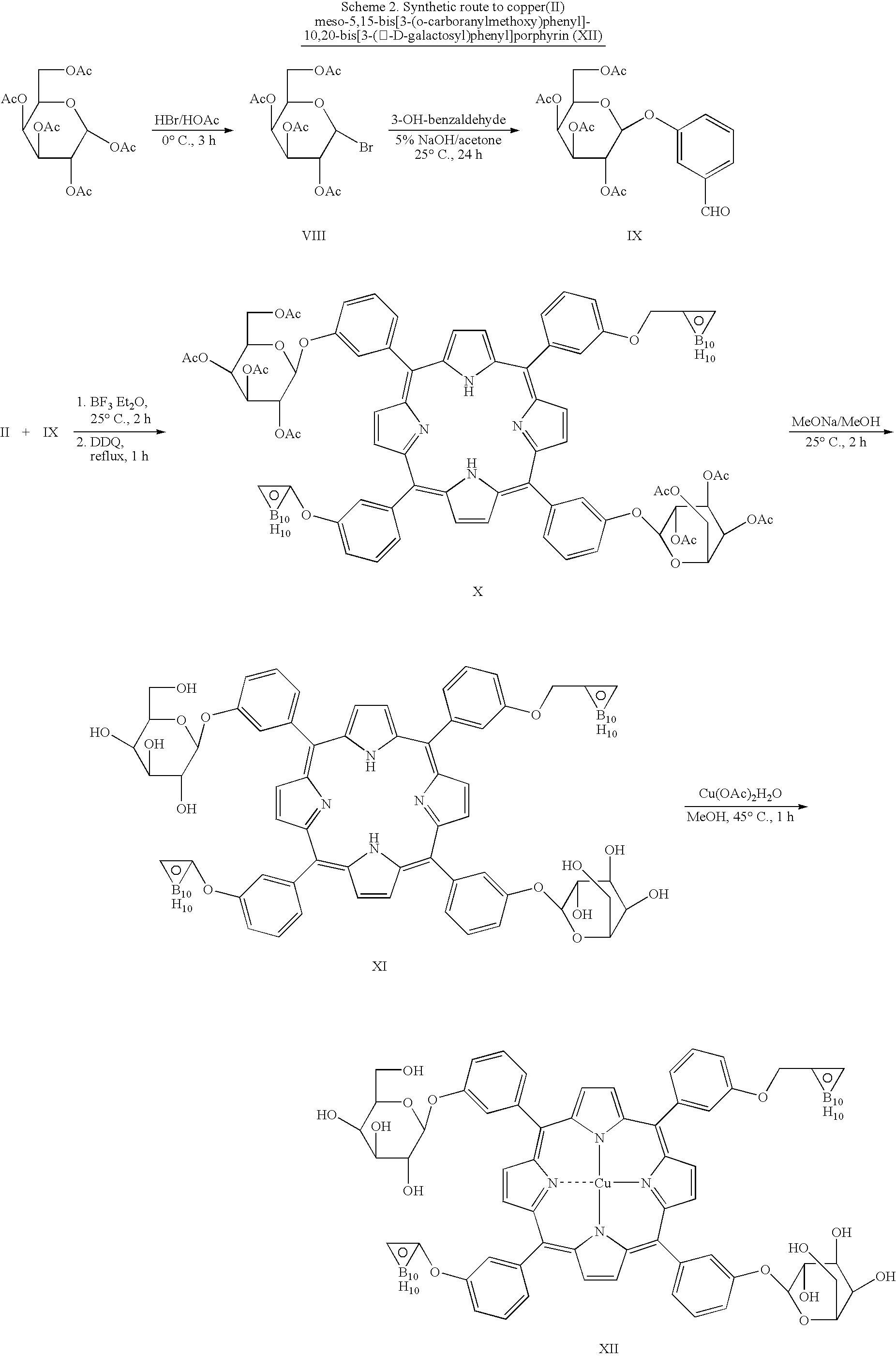

### Example 8

### Synthesis of α-D-Galactose tetraacetate bromide (VIII)

A method similar to that for synthesis of α-D-glucose tetraacetate bromide (III) was used. α-D-pentaacetyl galactose (7.8 g, 0.020 mol) gave 8.0 g product, 98%.

The product gave the following ¹H NMR spectrum in ppm (in CDCl₃, solvent): 1.91(s, 3H, CH₃), 1.96 (s, 3H, CH₃), 2.01 (s, 3H, CH₃), 2.05 (s, 3H, CH₃), 4.02-4.10 (m-d, 2H, CH₂), 4.40 (m, 1H), 4.94 (m, 1H), 5.28(m, 1H), 5.42 (m, 1H), 6.61 (s, 1H). The product gave the following proton-decoupled ¹³C NMR spectrum in ppm (in CDCl₃, solvent): 20.5 (CH₃), 20.5 (CH₃), 20.6 (CH₃), 60.8(CH₂), 66.9(CH), 67.7(CH), 67.9(CH), 71.1(CH), 88.3(CH), 169.6 (CO), 169.8 (CO), 169.9 (CO), 170.1 (CO).

### Example 9

### Synthesis of 3-(β-D-Galactose tetraacetate)benzaldehyde (IX)

A method similar to that for synthesis of 3-(β-D-glucosyl tetraacetate)benzaldehyde (IV) was used. α-D-galactose tetraacetate bromide (VIII) (8.0 g, 19 mmol) and 3-hydroxybenzaldehyde (3.7 g, 30 mmol) gave 4.3 g product, 50%.

The product gave the following ¹H NMR spectrum in ppm (in CDCl₃, solvent): 2.02(s, 3H, CH₃), 2.08 (d, 6H, CH₃), 2.19 (s, 3H, CH₃), 4.22 (s, 2H, CH₂), 5.21 (m, 2H, CH), 5.33 (s, 1H, CH), 5.51 (m, 2H, CH), 7.30 (m, 1H, ArH), 7.51 (m, 1H, ArH), 7.55 (m, 1H, ArH), 7.59 (m, 1H, ArH), 9.98 (s, 1H, CHO). The product gave the following proton-decoupled ¹³C NMR spectrum in ppm (in CDCl₃, solvent): 20.4 (CH₃), 20.5 (CH₃), 20.5 (CH₃), 20.6 (CH₃), 61.6 (CH₂), 67.0 (CH), 68.5 (CH), 70.6 (CH), 71.3 (CH), 98.9 (CH), 115.1 (ArC), 123.5 (ArC), 125.7 (ArC), 130.2 (ArC), 137.9 (ArC), 157.3 (ArC), 169.2 (CO), 169.9 (CO), 170.1 (CO), 170.4 (CO), 191.4 (CHO).

### Example 10

### Synthesis of meso-5,15-Bis[3-(o-carboranylmethoxy)phenyl]-10,20-bis[3-(β-D-galactose tetraacetate)phenyl]porphyrin (X)

A method similar to that for synthesis of meso-5,15-bis[3-(o-carboranylmethoxy)phenyl]-10,20-bis[3-(β-D-glucose tetraacetate)phenyl]porphyrin (V) was used. Reaction of 3-(β-D-galactose tetraacetate)benzaldehyde (IX) (450 mg, 1 mmol) and 3-(o-carboranylmethoxy)phenyl dipyrromethane (II) (397 mg, 1 mmol) gave 120 mg porphyrin, 15%.

The product gave the following ¹H NMR spectrum in ppm (in CDCl₃, solvent): −2.85 (s, 2 H, NH), 1.4-3.4 (20 H, carborane H), 2.00 (d, 12H, CH₃), 2.09 (s, 6H, CH₃), 2.15 (s, 6H, CH₃), 4.00 (1H, sugar, CH), 4.08 (s, 4H, CBCH), 4.56 (d, 6H, OCH₂), 5.17 (d, 2H, sugar CH), 5.32 (2H, sugar CH), 5.43 (m, 2H, sugar CH), 5.61 (m, 2H, sugar CH), 7.25 (m, 4H, ArH), 7.46(m, 1H, ArH), 7.68 (m, 8H, ArH), 7.90 (m, 6H, ArH), 8.85-8.90 (8H, pyr-H). The product gave the following proton-decoupled ¹³C NMR spectrum in ppm (in CDCl₃, solvent): 20.8 (sugar, CH₃), 20.8 (sugar, CH₃), 21.0 (sugar, CH₃), 21.0 (sugar, CH₃), 58.1 (CH₂), 61.6 (sugar CH₂), 67.1 (sugar CH), 68.9 (sugar CH), 69.6 (CBCCH), 71.0 (sugar CH), 71.4 (sugar CH), 71.6 (CBCCH), 100 (sugar CH), 108.3 (ArC), 114.4 (ArC), 117.8 (ArC), 119.5 (ArC), 119.9 (ArC), 121.3 (ArC), 128.0 (ArC), 128.2 (ArC), 129.3 (ArC), 130.0 (ArC), 143.6 (pyr), 143.9 (pyr), 155.6(ArC), 155.7 (ArC), 169.6 (CO), 170.3 (CO), 170.4 (CO), 170.4 (CO). The ultraviolet-visible absorbance spectrum of the product showed the following peaks in nanometers of wavelength (DCM): 418, 514, 550, 588, 645.

### Example 11

### Synthesis of meso-5,15-Bis[3-(o-carboranylmethoxy)phenyl]-10,20-bis[3-(¤-D-galactosyl)phenyl]porphyrin (XI)

A method similar to that for synthesis of meso-5,15-bis[3-(o-carboranylmethoxy)phenyl]-10,20-bis[3-(β-D-glucosyl)phenyl]porphyrin (VI) was used. 80 mg (0.0483 mmol) meso-5,15-bis[3-(o-carboranylmethoxy)phenyl]-10,20-bis[3-(β-D-galactose tetraacetate)phenyl]porphyrin (X) gave a red solid. 53 mg, 83%.

The product gave the following ¹H NMR spectrum in ppm (d6-DMSO, 2.50 ppm): −2.95 (s, 2 H, NH), 1.4-3.0 (m, 20H, carborane H), 3.35 (s, 10H, sugar OH), 3.35 (m, 2H, sugar CH), 3.57 (m, 4H, sugar CH), 3.70 (m, 2H, sugar CH), 4.50 (s, 4H, carborane CH₂), 4.55 (s, 2H, carborane CH), 4.84 (s, 4H, sugar CH₂), 5.16-5.29 (m, 2H, sugar CH), 5.39 (m, 2H, sugar, CH), 7.48 (m, 4H, ArH), 7.74 (m, 4H, ArH), 7.87 (m, 8H, ArH), 8.86 (8H, pyr-H). The product gave the following proton-decoupled ¹³C NMR spectrum in ppm (DMSO, 39.5 ppm): 55.8(sugar CH₂OH), 56.6 (sugar CHOH), 61.9 (CH₂C═CH), 68.5 (sugar CHOH), 68.7(sugar CHOH), 71 (—CH₂C═CH), 73.6(sugar CHOH), 75.5(sugar CHOH), 115 (ArC), 118-122 (ArC), 128 (ArC), 142.4 (pyr), 142.7 (pyr), 155.7 (ArC), 155.9 (ArC). The ultraviolet-visible absorbance spectrum of the product showed the following peaks in nanometers of wavelength (in DCM with trace DMSO): 419, 515, 551, 590, 644.

### Example 12

### Synthesis of Copper (II) meso-5,15-bis[3-(o-carboranylmethoxy)phenyl]-10,20-bis[3-(β-D-galactose)phenyl]porphyrin (XII)

A method similar to that for synthesis of copper (II) meso-5,15-bis[3-(o-carboranylmethoxy)phenyl]-10,20-bis[3-(¤-D-glucosyl)phenyl]porphyrin (VII) was used. Reaction of meso-5,15-bis[3-(o-carboranylmethoxy)phenyl]-10,20-bis[3-(¤-D-galactosyl)phenyl]porphyrin (XI) (53 mg, 0.040 mmol) gave 40 mg of copper (II) meso-5,15-bis[3-(o-carboranylmethoxy)phenyl]-10,20-bis[3-(β-D-galactose)phenyl]porphyrin (XII), 72%.

The ultraviolet-visible absorbance spectrum of the product showed the following peaks in nanometers of wavelength (in acetone solvent): 412, 537.

### C. Synthesis of copper (II) meso-5,15-bis[m-(3,5-di-o-carboranylmethoxybenzyloxy)phenyl]-10,20-bis[3-(β-D-glucosyl)phenyl]porphyrin

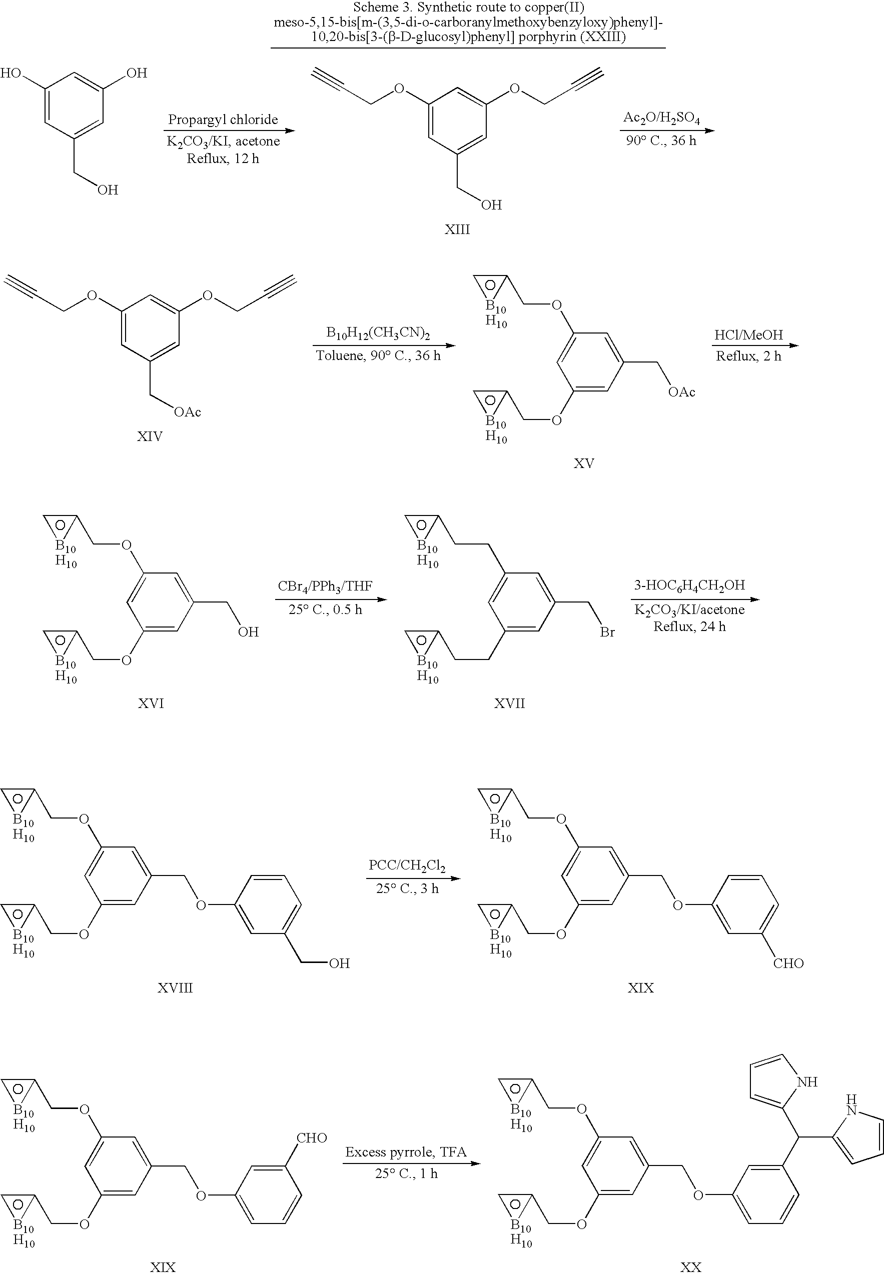

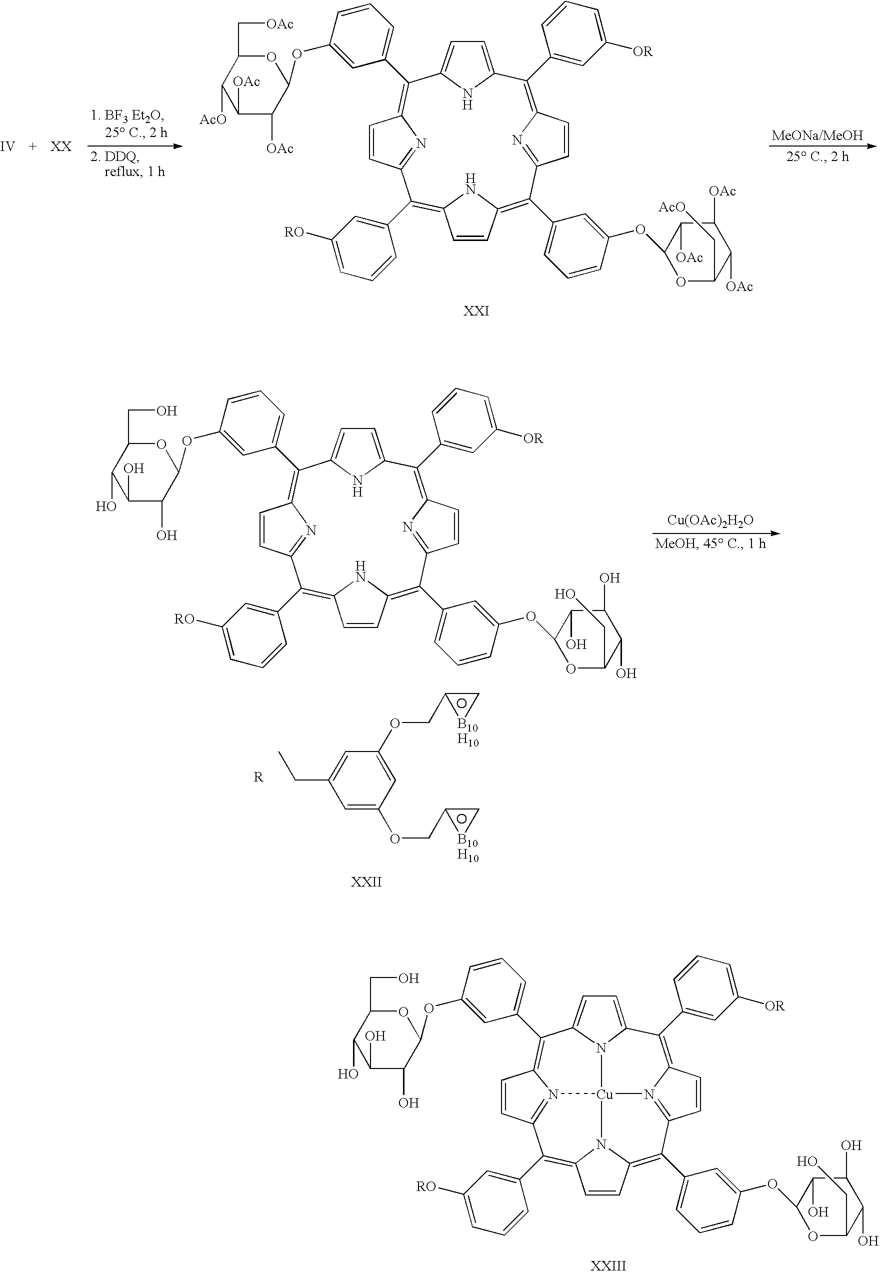

### Example 13

### Synthesis of 3,5-Dipropargyloxybenzyl alcohol (XIII)

Finely powdered K₂CO₃ (14 g, 0.10 mol), and KI (17 g, 0.10 mol) were stirred in acetone (200 mL) under a nitrogen atmosphere. 3,5-Dihydroxybenzyl alcohol (4.2 g, 0.030 mol), and propargyl chloride (5.3 g, 0.071 mol) were added and mixture was allowed to reflux overnight. After the solution was filtered, and evaporated down to dryness, the residue was diluted with DCM, worked up and the solvents were removed in vacuo leaving a yellow oil, which solidified upon standing to give 6.3 g in 97% yield.

The product had a melting point of 79-80° C. and gave the following ¹H NMR spectrum in ppm (in CDCl₃, solvent): 2.52 (t, 2H, C¤CH); 2.15 (br s, 1H, OH); 4.65 (d, 4H, ArOCH₂); 4.60 (s, 2H, ArH); 6.52 (s, 1H, ArH); 6.60 (s, 2H, ArH). The product gave the following proton-decoupled ¹³C NMR spectrum in ppm (in CDCl₃, solvent): 56.1 (ArOCH₂); 65.1 (ArCH₂); 75.9 (C¤C); 78.6 (C¤C); 101.6 (ArC); 106.4 (ArC); 143.8 (ArC); 159.0 (ArC).

C₁₃H₁₂O₃ requires 216.4, MS (FAB) m/e 217.5(M+H)⁺.

### Example 14

### Synthesis of 3,5-dipropargyloxybenzyl acetate (XIV)

3,5-Dipropargyloxybenzyl alcohol (XIII) (6.3 g, 0.029 mol) was stirred in acetic anhydride (7 mL, 0.07 mol). Concentrated sulfuric acid (2 drops) was added and the solution was stirred (90-100° C.) for 3 hours. The solution was then poured into ice water, neutralized with a saturated sodium carbonate solution and after the reaction was worked up, the product was purified using silica in a sintered glass funnel and the solvents were removed leaving a yellow oil, which solidified upon standing, 7.2 g in 96% yield.

The product had a melting point of 65-66° C. and gave the following ¹H NMR spectrum in ppm (in CDCl₃, solvent): 2.11 (s, 3H, CH₃); 2.54 (t, 2H, C¤CH); 4.67 (d, 4H, ArOCH₂); 5.05 (s, 2H, ArCH₂); 6.58 (s, 1H, ArH); 6.61 (s, 2H, ArH). The product gave the following proton-decoupled ¹³C NMR spectrum in ppm (in CDCl₃, solvent): 21.3 (CH₃); 56.0 (ArOCH₂); 66.2.(ArCH₂); 76.2 (C¤C); 78.6 (C¤C); 102.3 (ArC); 108.0 (ArC); 138.8 (ArC); 159.0 (ArC); 171.1 (CO).

C₁₅H₁₄O₄ requires 258.3, MS (FAB) m/e 259.5 (M+H)⁺.

### Example 15

### Synthesis of 3,5-Di-o-carboranylmethoxybenzyl acetate (XV)

Decaborane (2.70 g, 0.022 mol) was dissolved in dry toluene (80 mL) and stirred at room temperature under a nitrogen atmosphere. Anhydrous acetonitrile (12 mL, 0.22 mol) was added and the solution was allowed stirred for 3 hours. A solution of 3,5-dipropargyloxybenzyl acetate (XIV) (2.84 g, 0.011 mol) in toluene (80 mL) was added to the decaborane solution and the mixture was heated to 80-90° C. and was maintained at this temperature for 3 days, after which time the results from TLC showed no presence of starting material. The excess decaborane was decomposed by the slow addition of methanol (20 mL) while cooling in an ice-water bath. After the solvents were removed by rotary evaporation the resulting residue was dissolved in DCM, washed with 10% sodium bicarbonate, water, dried over anhydrous sodium sulfate and the solvent was removed by rotary evaporation, leaving a yellow oil which crystallized upon standing yielding 4.40 g in 81% yield.

The product had a melting point of 122-123° C. and gave the following ¹H NMR spectrum in ppm (in CDCl₃, solvent): 1.4-3.4 (m, 20H, carborane H), 2.12 (s, 3H, CH₃); 4.06 (s, 2H, CCHB₁₀H₁₀); 4.39 (s, 4H, ArOCH₂); 5.01 (s, 2H, ArCH₂); 6.32 (s, 1H, ArH); 6.52 (s, 2H, ArH). The product gave the following proton-decoupled ¹³C NMR spectrum in ppm (in CDCl₃, solvent): 21.4 (CH₃); 58.3 (ArOCH₂); 65.8 (ArCH₂); 69.6 (—CCHB₁₀H₁₀); 71.5 (—CCHB₁₀H₁₀); 102.3 (ArC); 108.5 (ArC); 139.8 (ArC); 158.6 (ArC); 171.0 (CO). C₁₅H₃₄O₂₀ requires 494.6, MS (FAB) m/e 496.0(M+H)⁺.

### Example 16

### Synthesis of 3,5-Di-o-carboranylmethoxybenzyl alcohol (XVI)

Concentrated HCl (4.0 mL) was added to a solution of 3,5-di-o-carboranylmethoxybenzyl acetate (XV) (4.0 g, 8.0 mmol) in methanol (60 mL) and the mixture was allowed to stir at reflux for 2 hours, after which time TLC showed no starting material. The solvents were then removed leaving a yellow oil, which solidified to a white solid upon standing, 3.4 g, 93% yield.

The product had a melting point of 267-269° C. and gave the following ¹H NMR spectrum in ppm (in CDCl₃, solvent): 1.4-3.4 (m, 20H, carborane H), 2.54 (br s, 1H, OH); 4.04 (s, 2H, CCHB₁₀H₁₀); 4.40 (s, 4H, ArOCH₂); 4.65 (s, 2H, ArCH₂); 6.28 (s, 1H, ArH); 6.54 (s, 2H, ArH). ¹³C NMR (CDCl₃): 58.0 (ArOCH₂); 64.7 (ArCH₂); 69.4 (—CCHB₁₀H₁₀); 71.3 (—CCHB₁₀H₁₀); 101.5 (ArC); 106.6 (ArC); 144.7 (ArC); 158.5 (ArC). C₁₃H₃₂O₂₀ requires 452.6, MS (FAB) m/e 453.0 (M⁺).

### Example 17

### Synthesis of 3,5-Di-o-carboranylmethoxybenzylbromide (XVII)

To a solution of 3,5-di-o-carboranylmethoxybenzyl alcohol (XVI) (0.454 g, 1.0 mmol) and carbon tetrabromide (0.398 g, 1.2 mmol) in a minimal amount of dry THF (˜2 mL), triphenylphosphine (0.314 g, 1.2 mmol) was added and the mixture was stirred under argon for 20 min. The reaction mixture was then poured into water and the product extracted with DCM (3×7 mL). The combined extracts were dried with K₂CO₃ and then purified using a silica pad washed with DCM. The solution was evaporated to dryness yielding a white solid 0.485 g, 92%.

The product had a melting point of 230-232° C. and gave the following ¹H NMR spectrum in ppm (in CDCl₃, solvent): 1.4-3.4 (m, 20H, carborane H), 4.02 (s, 2H, CH), 4.37 (s, 2H, CH₂Br), 4.39 (s, 4H, ArOCH₂), 6.26 (s, 1H, ArH), 6.55 (s, 2H, ArH). ¹³C NMR: δ 32.4 (CH₂Br), 58.0 (ArOCH₂), 69.5 (—CCHB₁₀H₁₀), 71.1 (—CCHB₁₀H₁₀), 102.3 (ArC), 109.2 (ArC), 141.2 (ArC), and 158.4 (ArC). C₁₃H₃₁O₂B₂₀Br requires 515.5, MS (FAB) m/e 516.9 (M+H)⁺.

### Example 18

### Synthesis of 3-(3,5-Di-o-carboranylmethoxybenzyloxy)benzylalcohol (XVIII)

K₂CO₃ (0.210 g, 1.5 mmol) and KI (0.25 g, 1.5 mmol) were placed in a 50 mL round-bottom flask 3,5-di-o-carboranylmethoxybenzylbromide (XVII) (0.410 g, 0.80 mmol), 3-hydroxybenzylalcohol (0.100 g, 0.80 mmol) were dissolved in acetone (20 mL). Under argon the mixture was refluxed for 24 hours. The solvents were evaporated; the residue was extracted with DC, and worked up yielding a white solid, 0.430 g, 96%.

The product had a melting point of 259-261° C. and gave the following ¹H NMR spectrum in ppm (in CDCl₃, solvent): 1.70 (s, 1H, OH), 1.4-3.4 (m, 20H, carborane H), 4.04 (s, 2H, CH), 4.40 (s, 4H, CH₂CCHB₁₀H₁₀), 4.67 (s, 2H, ArCH₂OH), 5.00 (s, 2H, ArCH₂OAr), 6.31 (s, 1H, ArH), 6.60 (s, 2H, ArH), 6.87 (m, 1H, ArH), 7.00 (m, 2H, ArH), and 7.26 (m, 1H, ArH). The product gave the following proton-decoupled ¹³C NMR spectrum in ppm (in CDCl₃, solvent): 58.2 (CH₂CCHB₁₀H₁₀), 65.5 (ArCH₂ OH), 69.6 (—CCHB₁₀H₁₀), 71.4 (—CCHB₁₀H₁₀), 102.0 (ArC), 107.4 (ArC), 113.6 (ArC), 114.3 (ArC), 120.2 (ArC), 130.2 (ArC), 141.2 (ArC), 143.1 (ArC), 158.7 (ArC), and 158.8 (ArC). C₂₀H₃₈O₄B₂₀ requires 558.7, MS (FAB) m/e 559.0 (M⁺).

### Example 19

### Synthesis of 3-(3,5-Di-o-carboranylmethoxybenzyloxy)benzaldehyde (IXX)

Pyridinum chlorochromate (PCC) (0.172 g, 0.80 mmol) was dissolved in 10 mL DCM and cooled in ice water bath. 3-(3,5-Di-o-carboranylmethoxybenzyloxy)benzyl alcohol (XVIII) (0.223 g, 0.40 mmol), dissolved in 10 mL DCM, was added dropwise to the PCC solution. The mixture was stirred for 2 hours. TLC showed only one new compound. The heterogeneous solution is filtered through a sintered glass funnel containing silica (2 cm). The flask and the silica were washed thoroughly with excess DCM. The solvents were removed, yielding a white solid 0.220 g, 99%.

The product had a melting point of 263-265° C. and gave the following ¹H NMR spectrum in ppm (in CDCl₃, solvent): 1.4-3.4 (m, 20H, carborane H), 4.04 (s, 2H, —CCHB₁₀H₁₀), 4.42(s, 4H, ArOCH₂), 5.00 (s, 2H, ArCH₂O), 6.33 (s, 1H, ArH), 6.61 (s, 2H, ArH), 7.23 (s, 1H, ArH), 7.44 (m, 1H, ArH), 7.50 (m, 2H, ArH), and 9.98 (s, 1H, CHO). The product gave the following proton-decoupled ¹³C NMR spectrum in ppm (in CDCl₃, solvent): 58.0 (CH₂CCHB₁₀H₁₀), 69.5 (—CCHB₁₀H₁₀), 69.7 (ArCH₂OAr), 71.2 (—CCHB₁₀H₁₀), 102.0 (ArC), 107.4 (ArC), 112.8 (ArC), 122.4 (ArC), 124.8 (ArC), 130.6 (ArC), 138.1 (ArC), 138.4 (ArC), 140.3 (ArC), 158.6 (ArC), and 192.1(CHO). C₂₀H₃₆O₄B₂₀ requires 556.7, MS (FAB) m/e 558.0 (M+H)⁺.

### Example 20

### Synthesis of 3-(3,5-Di-o-carboranylmethoxybenzyloxy)phenyl dipyrromethane (XX)

A method similar to that for the synthesis of 3-(o-carboranylmethoxy) phenyldipyrromethane (II) was used. Excess freshly distilled pyrrole (34 mL, 0.50 mol) and 3-(3,5-di-o-carboranylmethoxybenzyloxy)benzaldehyde (IXX) (2.8 g, 5.0 mmol) were used to obtain 3.1 g product, corresponding to a yield of 93%.

The product gave the following ¹H NMR spectrum in ppm (in CDCl₃, solvent): 1.4-3.4 (m, 20H, carborane H), 4.03 (s, 1H, —CCHB₁₀H₁₀), 4.34 (s, 2H, B₁₀H₁₀CH₂), 5.42 (s, 1H, ArCH), 5.89 (m, 1H, ¤-pyr-H), 6.16 (m, 1H, ¤-pyr-H), 6.70 (m, 2H, ¤-pyr-H), 6.70 (m, 2H, ArH), 6.73 (m, 1H, ArH), 6.89 (m, 1H, ArH), 7.24 (m, ArH), 7.90 (ArH). The product gave the following proton-decoupled ¹³C NMR spectrum in ppm (in CDCl₃, solvent): 44.0 (CH), 58.0 (B₁₀H₁₀CH₂), 69.2 (—CCHB₁₀H₁₀), 69.3 (ArCH₂), 71.8 (—CCHB₁₀H₁₀), 101.9 (ArC), 107.3 (pyr), 108.4 (pyr), 113.1 (ArC), 115.2(ArC), 117.5(pyr), 121.6 (ArC), 129.8 (AC), 132.3(pyr), 140.6 (ArC), 144.0 (ArC), 158.3 (ArC), 158.6 (ArC). C₂₈H₄₆N₂O₃B₂₀ requires 672.9, MS (FAB) m/e 672.5 (M⁺).

### Example 21

### Synthesis of meso-5,15-bis[m-(3,5-di-o-carboranylmethoxybenzyloxy)-phenyl]-10,20-bis[3-(β-D-glucose tetraacetate)phenyl]porphyrin (XXI)

A method similar to that for the synthesis of meso-5,15-bis[3-(o-carboranylmethoxy)phenyl]-10,20-bis[3-(β-D-glucose tetraacetate)phenyl]porphyrin (V) was used. Reaction of 3-(β-D-glucosyl tetraacetate)benzaldehyde (IV) (452 mg, 1.0 mmol) and 3-(3,5-di-o-carboranylmethoxybenzyloxy)phenyl dipyrromethane (XX) (677 mg, 1.0 mmol) gave 133 mg red solid, 12%.

The product gave the following ¹H NMR spectrum in ppm (CDCl₃, 7.26 ppm): −2.80 (s, 2H, NH), 1.99 (CH₃), 2.03(CH₃), 2.08(CH₃), 1.4-3.4 (m, 40H, Carborane H), 3.75(m, 2H, sugar H), 3.98(4H, —CCHB₁₀H₁₀), 4.14 (m, 2H, sugar H), 4.36 (s, 8H, B₁₀H₁₀CH₂), 4.58(m, 2H, sugar H), 5.19 (s, 4H, ArCH₂), 5.38 (m, 2H, sugar H), 6.31(m, 4H, ArH), 6.67(m, 8H, ArH), 7.41 (m,4H, ArH), 7.67-7.70 (m, 4H, ArH), 7.69 (m, 4H, ArH), 7.85 (m, 8H, ArH), 8.86 (d, 8H, PyH). ¹³C NMR: 20.2 (sugar CH₃), 20.7 (sugar CH₃), 20.8 (sugar CH₃), 20.9 (sugar CH₃), 58.1 (B₁₀H₁₀CH₂), 62.2 (CH₂), 68.4 (sugar CH), 69.4 (—CCHB₁₀H₁₀), 69.7 (ArCH₂), 71.3 (—CCHB₁₀H₁₀), 71.4 (sugar CH), 72.4 (sugar CH), 72.9 (sugar CH), 99.5 (sugar CH), 101.9 (ArC), 107.4 (ArC), 114.6 (ArC), 116.9 (ACr), 119.6 (ArC), 120.0 (ArC), 121.7 (ArC), 128.1 ArC), 128.4(ArC), 130.0 (ArC), 140.8 (pyr), 143.6 (ArC), 143.7 (ArC), 155.6 (ArC), 156.9 (ArC), 158.6 (ArC), 169.6 (CO), 169.6 (CO), 170.4 (CO), 170.6 (CO). The ultraviolet-visible absorbance spectrum of the product showed the following peaks in nanometers of wavelength (DCM): 420, 422, 515, 550, 590, 646. C₉₈H₁₃₀O₂₆N₄B₄₀ requires 2212.5, MS (FAB) m/e 2211.0 (M−H)⁺.

### Example 22

### Synthesis of meso-5,15-bis[m-(3,5-di-o-carboranylmethoxybenzyloxy)phenyl]-10,20-bis[3-(β-D-glucosyl)phenyl]porphyrin (XXII)

A method similar to that for the synthesis of meso-5,15-bis[3-(o-carboranylmethoxy)phenyl]-10,20-bis[3-(β-D-glucosyl)phenyl]porphyrin (VI) was used. 111 mg (0.050 mmol) meso-5,15-bis[m-(3,5-di-o-carboranylmethoxybenzyloxy)phenyl]-10,20-bis[3-(β-D-glucose tetraacetate)phenyl]porphyrin (XXI) gave 78 mg of a red solid, yielding 83%.

The product gave the following ¹H NMR spectrum in ppm (CDCl₃, 7.26 ppm): −2.80 (s, 2H, NH), 1.4-3.4 (m, 40H, carborane CH), 3.75 m, 2H, sugar H), 3.98 (4H, —CCHB₁₀H_{10,}), 4.14 (m, 2H, sugar H), 4.36 (s, 8H, B₁₀H₁₀CH₂), 4.58(m, 2H, sugar H), 5.19 (s, 4H, ArCH₂), 5.38 (m, 2H, sugar H), 6.31 m, 4H, ArH), 6.67 (m, 8H, ArH), 7.41 (m, 4H, ArH), 7.67-7.70 (m, 4H, ArH), 7.69 (m, 4H, ArH), 7.85 (m, 8H, ArH), 8.86 (d, 8H, PyH). The product gave the following ¹H NMR spectrum in ppm (DMSO, 2.50 ppm): −2.89 (s, 2H, NH), 1.4-3.4 (m, 40H, carborane H), 3.28 (m, 2H, sugar H), 3.40 m, 6H, sugar OH), 3.74 (m, 2H, sugar H), 3.98 (4H, —CCHB₁₀H_{10,}), 4.14 (m, 2H, sugar H), 4.36 (s, 8H, B₁₀H₁₀CH₂), 4.61 (s, 4H, ArCH₂), 5.29 (s, 4H, ArCH₂), 5.38 (m, 2H, sugar H), 6.67 (m, 4H, ArH), 6.87 (m, 8H, ArH), 7.52-7.59 (m, 4H, ArH), 7.76 (m, 4H, ArH), 7.84 (m, 4H, ArH), 7.92 (m, 4H, ArH), 8.92 (d, 8H, PyH). The product gave the following proton-decoupled ¹³C NMR spectrum in ppm (DMSO, 2.50 ppm): 54.9 (sugar CH₂), 55.8 (sugar CH), 60.7 (sugar CH), 61.8 (B₁₀H₁₀CH₂), 68.7 (—CCHB₁₀H₁₀), 69.1 (ArCH₂), 69.7 sugar CH), 73.3 (CHB₁₀H₁₀), 73.4 (sugar CH), 76.6 (sugar CH), 77.0 (sugar CH), 100.5 (Ar), 101.3 (Ar), 107.7 (Ar), 114.6 (Ar), 119.7 (Ar), 121.2 (Ar), 127.5 (Ar), 128.0 (Ar), 139.9 (Ar), 142.4 (Pyr), 142.6 (Pyr), 155.9 (Ar), 156.7 (Ar), 158.3 (Ar), 208.3 (DMSO). The ultraviolet-visible absorbance spectrum of the product showed the following peaks in nanometers of wavelength (DCM): 420, 515, 551, 590, 645.

### Example 23

### Synthesis of copper (II) meso-5,15-bis[m-(3,5-di-o-carboranylmethoxybenzyloxy)phenyl]-10,20-bis[3-(β-D-glucosyl)phenyl]porphyrin (XXIII)

A method similar to that for the synthesis of copper (II) meso-5,15-bis[3-(o-carboranylmethoxy)phenyl]-10,20-bis[3-(β-D-glucosyl)phenyl]porphyrin (VII) was used. Reaction of meso-5,15-bis[m-(3,5-di-o-carboranylmethoxybenzyloxy)phenyl]-10,20-bis[3-(β-D-glucosyl)phenyl]porphyrin (XXII) (70 mg, 0.037 mm) and copper acetate monohydrate (10 mg, 0.050 mmol) gave 54 mg product, 75%.

The ultraviolet-visible absorbance spectrum of the product showed the following peaks in nanometers of wavelength (in acetone solvent): 415, 541.

### Example 24

### Preparation of boronated porphyrin solutions

Porphyrin compounds (VII, XII, XXIII) were emulsified in 3% Cremophor EL and 6% propylene glycol in saline.

To prepare a solution of ˜3.5 mg/mL porphyrin in 3% Cremophor EL (CRM) and 6% propylene glycol (PRG), the porphyrin was dissolved in tetrahydrofuran (THF) (1.5% of the total volume) and then heated to 40° C. for 15 min. CRM (3% of total volume) was then added and the mixture was heated to 60° C. for 2 hours, which removed most of the THF. After cooling to room temperature, PRG (6% of total volume) was added, followed by slow dropwise addition of saline (89.5% of total volume) with rapid stirring. The solution was degassed by stirring under vacuum (˜30 mm Hg) for 30-60 min and then filtered (Millipore, 8 μm).

### Example 25

### Biodistribution of porphyrins VII and XII in mice bearing EMT-6 carcinomas

BALB/c mice bearing subcutaneously implanted EMT-6 mammary carcinomas implanted on the dorsal thorax were given a total dose of 200-230 milligrams porphyrin compound (VII or XII) per kilogram body weight (32-37 mg B/kg). Mice were given six intraperitoneal injections over a 2-day period with 3 injections per day at 4-hour intervals. At one and two days after the last injection, mice were euthanized, and tumor, blood, brain, and liver were removed for boron analyses by direct current-plasma-atomic-emission spectrometry (DCP-AES). The blood was first analyzed for hematologic parameters that indicate toxicity before it was analyzed for boron. Table 1 shows the average boron concentrations for different types of tissue from BALB/c mice.

TABLE 1
Average boron concentrations (μg/g wet tissue) in various tissues in mice
(n = 5) given 200 mg/kg porphyrin VII or 230 mg/kg XII in 6 i.p. injections over a
period of 2 days at either 1 or 2 days after the last injection.

| | | Time after | | | | |
|---|---|---|---|---|---|---|
| | Porphyrin | last | EMT-6 | | | |
| | dose/boron | injection | Tumor | Blood | Brain | Liver |
| Porphyrin | dose (μg/gbw) | (d) | (μg B/g) | (μg B/g) | (μg B/g) | (μg B/g) |
| VII | 200/32 | 1 | 20.2 ± 6.3 | 0.4 ± 0.1 | 0.0 ± 0.0 | 457 ± 74 |
| VII | 200/32 | 2 | 19.0 ± 5.4 | 0.2 ± 0.0 | 0.0 ± 0.0 | 492 ± 83 |
| XII | 230/37 | 1 | 16.7 ± 8.3 | 0.4 ± 0.1 | 0.0 ± 0.1 | 473 ± 102 |
| XII | 230/37 | 2 | 12.6 ± 5.2 | 0.2 ± 0.1 | 0.0 ± 0.1 | 354 ± 352 |

The results of the preliminary biodistribution study showed that the tumor boron concentrations are adequate for therapy. The tumor to blood boron ratios for Porphyrin VII are greater than 50:1 and the ratios for Porphyrin XII are greater than 40:1. Extremely small amounts of boron were found in the brain. Such data indicate that BNCT may be effective in the treatment of cancers of the head and neck or brain.

### Example 26

### Weight changes and hematologic parameters from porphyrins VII and XII

TABLE 2
Weight changes and hematologic parameters in mice given 200 mg/kg
porphyrin VII or 230 mg/kg porphyrin XII compared with untreated controls at 1 or 2
days after the last injection. Values are reported as median (and range).

| | Time | | | | | |
|---|---|---|---|---|---|---|
| | after last | | | | | |
| | injection | No. of | % Weight | Platelets | Lymphocytes | Granulocytes |
| Porphyrin | (d) | mice | change | (10³/mm³) | (% WBC) | (% WBC) |
| VII | 1 | 5 | −0.18 (−5.6-4.7) | 504 (111-783)* | 47 (37-54) | 49 (43-61) |
| VII | 2 | 5 | 3.5 (−1.5-3.9) | 935 (840-1039) | 52 (44-56) | 45 (40-54) |
| XII | 1 | 5 | 1.2 (−3.5-4.8) | 962 (944-1306) | 36 (26-59) | 61 (38-71) |
| XII | 2 | 5 | 3.4 (−2.2-4.8) | 797 (736-877) | 54 (22-63) | 43 (34-76) |
| Controls | 1 | 4 | | 1127 (1054-1208) | 51 (49-54) | 46 (44-48) |
| Controls | 2 | 3 | | 898 (831-916) | 32 (29-66) | 65 (28-68) |

*The non-parametric Wilcoxon two-sample test with the corresponding untreated group shows that P < 0.05

Hematological data showed that the porphyrin-treated groups were not significantly different from age-matched controls except the platelet count from porphyrin VII one day after the last injection. However, even this slight abnormality diminished after one more day. At necropsy, all tissues appeared normal. Hematological data, particularly the platelet count and the weight data indicate that the dose can be escalated without increasing the toxicity.

### Example 27

### Biodistribution of Porphyrins VII and XXIII in mice bearing SCCVII squamous cell carcinomas

C3H mice bearing subcutaneously implanted SCCVII squamous cell carcinomas implanted on the dorsal thorax were given a total dose of 450 milligrams porphyrin compound VII per kilogram body weight (70 mg B/kg) via 6 i.p. injections given over a period of 2 days (3/day over 8 hours). Mice were given porphyrin XXIII at a total dose of 160 mg/kg (36 mg/kg B) via 3 i.p. injections given over a period of 8 hours. At one and two days after the last injection, mice were euthanized, and tumor, blood, brain, and liver were removed for boron analyses. The blood was first analyzed for hematologic parameters that indicate toxicity before it was analyzed for boron. Table 3 shows the average boron concentrations for different types of tissue from C3H mice.

TABLE 3
Average boron concentrations (μg/g wet tissue) in various tissues in mice
(n = 4 or 5) given 450 or 160 mg/kg porphyrin VII or XXIII (70 or 36 mg B/kg,
respectively) in 6 or 3 i.p. injections over 2 or 1 day, respectively, at either 1 or 2 days
after the last injection.

| | Porphyrin | Time | | | | | |
|---|---|---|---|---|---|---|---|
| | dose/boron | after last | | SCCVII | | | |
| | dose | injection | Number | Tumor | Blood | Brain | Liver |
| Porphyrin | (μg/gbw) | (d) | of mice | (μg/g) | (μg/g) | (μg/g) | (μg/g) |
| VII | 450/70 | 1 | 5 | 29.1 ± 7.9 | 1.0 ± 0.1 | 0.3 ± 0.1 | 787 ± 70 |
| XXIII | 160/39 | 1 | 4 | 16.9 ± 2.6 | 0.6 ± 0.1 | 0.1 ± 0.0 | 540 ± 32 |
| XXIII | 160/39 | 2 | 4 | 14.5 ± 2.9 | 0.3 ± 0 | 0.0 ± 0.0 | 534 ± 59 |

### Example 28

### Weight changes and hematologic parameters from porphyrins VII and XXIII

TABLE 4
Weight changes and hematologic parameters in mice given 450 or 180 mg/kg
porphyrin VII or XXIII (70 or 39 mg B/kg, respectively) or untreated controls at
1 or 2 days after the last injection. Values are reported as median (and range).

| | Time | | | | | |
|---|---|---|---|---|---|---|
| | after last | | | | | |
| | injection | Number | % Weight | Platelets | Lymphocytes | Granulocytes |
| Compound | (d) | of mice | change | (10³/mm³) | (% WBC) | (% WBC) |
| VII | 1 | 5 | −1.2 (−2.6-1.9) | 739 (628-751) | 50 (46-56) | 46 (41-50) |
| XXIII | 1 | 4 | 0.5 (−1.9-3.2) | 775 (618-881) | 53 (48-64) | 43 (32-46) |
| XXIII | 2 | 4 | 3.2 (0.9-6.3) | 655 (610-723) | 57 (51-59) | 39 (36-43) |
| Controls | 1 | 4 | | 822 (675-945) | 61 (55-63) | 34 (33-41) |
| Controls | 2 | 4 | | 876 (818-911) | 61 (59-72) | 34 (24-36) |

Even at the very high dose of 450 mg/kg, mice given porphyrin VII showed no thrombocytopenia. Mice given porphyrin XXIII at a moderately high dose of 160 mg/kg, also did not show any decrease in platelet count in comparison with the control mice. Toxicity appears to be minimal to non-existent judging by the very low weight loss.

Thus, while there have been described the preferred embodiments of the present invention, those skilled in the art will realize that other embodiments can be made without departing from the spirit of the invention, which includes all such further modifications and changes as come within the true scope of the claims set forth herein.

1. A compound of the formula 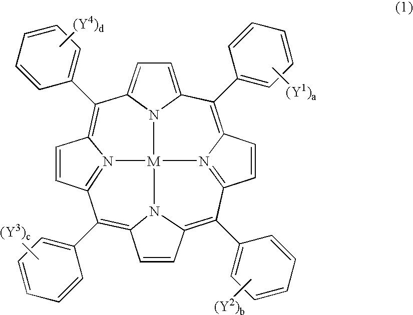
wherein:
at least one of Y¹, Y², Y³, and Y⁴ represents a mono-saccharide, di-saccharide, tri-saccharide, or oligo-saccharide and at least one of Y¹, Y², Y³, and Y⁴ is a substituent represented by formula (2)
—X¹—(CR¹R²)ᵣ-Z (2)
wherein Z is a carborane cluster or a substituent represented by formula (3): 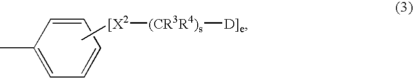
wherein D is a carborane cluster;
Y¹, Y², Y³, and Y⁴ are independently on either or both of the ortho and/or meta positions or on the para position on the phenyl rings;
the Y¹, Y², Y³, and Y⁴ that are not a mono-saccharide, di-saccharide, tri-saccharide, or oligo-saccharide and that are not represented by formula (2) or (3) are independently hydrogen, a hydrocarbyl, non-aromatic carbocyclic, non-aromatic heterocyclic, aryl, alkylaryl, or arylalkyl group substituted with 1 to 4 hydrophilic groups selected from hydroxy, alkoxy, —C(O)OR⁵, —SOR⁶, —SO₂R⁶, nitro, amido, ureido, carbamato, —SR⁷, —NR⁸R⁹, or poly-alkyleneoxide;
X¹ and X² are independently oxygen or sulfur;
R¹, R², R³, R⁴, R⁵, R⁶, R⁷, R⁸, and R⁹ are independently selected from hydrogen and C₁ to C₄ hydrocarbyl;
r and s are independently 0 or an integer from 1 to 20;
a, b, c, d, and e independently represent an integer from 1 to 4; and
M is either two hydrogen ions, a single monovalent metal ion, two monovalent metal ions, a divalent metal ion, a trivalent metal ion, a tetravalent metal ion, a pentavalent metal ion, a hexavalent metal ion wherein the porphyrin-metal complex derived from a single monovalent metal ion is charge-balanced by a counter cation, and the porphyrin-metal complex derived from a trivalent, tetravalent, pentavalent, hexavalent metal ion is charge-balanced by an appropriate number of counter anions, dianions, or trianions.

2. The compound according to claim 1 wherein Z is selected from the carborane clusters —C₂HB₉H₁₀ or —C₂HB₁₀H₁₀, wherein —C₂HB₉H₁₀ is nido ortho-, meta-, or para-carborane, and —C₂HB₁₀H₁₀ is closo ortho-, meta-, or para-carborane.

3. The compound according to claim 1 wherein Z is represented by the formula (3) and D is selected from the carborane clusters —C₂HB₉H₁₀ or —C₂HB₁₀H₁₀, wherein —C₂HB₉H₁₀ is nido ortho-, meta-, or para-carborane, and —C₂HB₁₀H₁₀ is closo ortho-, meta-, or para-carborane.

4. The compound according to claim 1 wherein M is a metal ion selected from the group consisting of a radioactive metal ion useful in radioisotope-mediated radiation therapy, a radioactive metal ion imageable by single photon emission computed tomography (SPECT) or positron emission tomography (PET), a paramagnetic metal ion detectable by magnetic resonance imaging (MRI), a metal ion suitable for boron neutron capture therapy (BNCT) or photodynamic therapy (PDT), or a combination thereof.

5. The compound according to claim 4, wherein M is selected from the group consisting of vanadium, manganese, iron, ruthenium, technetium, chromium, platinum, cobalt, nickel, copper, zinc, germanium, indium, tin, yttrium, gold, barium, tungsten, gadolinium, or a combination thereof.

6. The compound according to claim 1, wherein at least one of Y¹, Y², Y³, and Y⁴ represents a mono-saccharide.

7. The compound according to claim 6, wherein the mono-saccharide is selected from the group consisting of D or L, alpha and beta forms of glucose, fructose, galactose, mannose, gulose, ribose, arabinose, xylose, and ribulose.

8. The compound according to claim 7, wherein the mono-saccharide is selected from the group consisting of D or L, alpha and beta forms of glucose and galactose.

9. The compound according to claim 1, wherein at least one of Y¹, Y², Y³, and Y⁴ represents a di-saccharide.

10. The compound according to claim 9, wherein the di-saccharide is selected from the group consisting of D or L, alpha and beta forms of sucrose, maltose, and lactose.

11. The compound according to claim 1 wherein
i. a, b, c, and d are 1;
ii. two of Y¹, Y², Y³ and Y⁴ are represented by formula (2); and
iii. the two of Y¹-Y⁴ not represented by formula (2) are a mono-saccharide, di-saccharide, tri-saccharide, or oligo-saccharide.

12. The compound according to claim 11 wherein the substituents represented by formula (2) are in the cis configuration.

13. The compound according to claim 11 wherein the substituents represented by formula (2) are in the trans configuration.

14. The compound according to claim 11 wherein the mono-saccharide, is selected from the group consisting of D or L, alpha and beta forms of glucose and galactose.

15. The compound according to claim 11, wherein M is selected from the group consisting of vanadium, manganese, iron, ruthenium, technetium, chromium, platinum, cobalt, nickel, copper, zinc, germanium, indium, tin, yttrium, gold, barium, tungsten, gadolinium or combination thereof.

16. The compound according to claim 11, wherein X¹ is oxygen; R¹ and R² are hydrogen; and r is 1.

17. The compound according to claim 11 wherein Z is selected from the carborane clusters —C₂HB₉H₁₀ or —C₂HB₁₀H₁₀, wherein —C₂HB₉H₁₀ is nido ortho-, meta-, or para-carborane, and —C₂HB₁₀H₁₀ is closo ortho-, meta-, or para-carborane.

18. The compound according to claim 17 wherein the substituents represented by formula (2) are on the meta position of each phenyl ring.

19. The compound according to claim 11 wherein Z is represented by formula (3) and D is selected from the carborane clusters —C₂HB₉H₁₀ or —C₂HB₁₀H₁₀, wherein —C₂HB₉H₁₀ is nido ortho-, meta-, or para-carborane, and —C₂HB₁₀H₁₀ is closo ortho-, meta-, or para-carborane.

20. The compound according to claim 19 wherein X² is oxygen, R³ and R⁴ are hydrogen, s is 1, and e is 2.

21. The compound according to claim 20 wherein the structures —X²—(CR³R⁴)ₛ-D are in the 3 and 5 positions of each phenyl ring.

22. A method of imaging a tumor and surrounding tissue in a subject comprising:
administering to the subject a composition comprising a compound selected from the group consisting of:
a) a compound of the formula 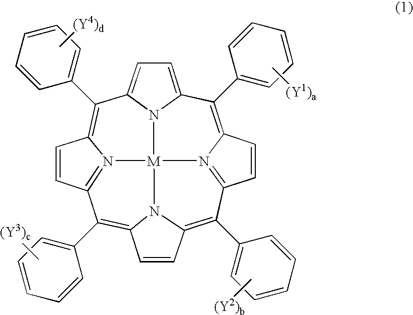
wherein:
at least one of Y¹, Y², Y³, and Y⁴ represents a mono-saccharide, di-saccharide, tri-saccharide, or oligo-saccharide and at least one of Y¹, Y², Y³, and Y⁴ is a substituent represented by formula (2)
—X¹—(CR¹R²)ᵣ-Z (2)
wherein Z is a carborane cluster or a substituent represented by formula (3): 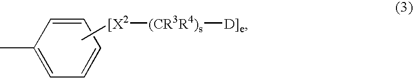
wherein D is a carborane cluster;
Y¹, Y², Y³, and Y⁴ are independently on either or both of the ortho and/or meta positions or on the para position on the phenyl rings;
the Y¹, Y², Y³, and Y⁴ that are not a mono-saccharide, di-saccharide, tri-saccharide, or oligo-saccharide and that are not represented by formula (2) or (3) are independently hydrogen, a hydrocarbyl, non-aromatic carbocyclic, non-aromatic heterocyclic, aryl, alkylaryl, or arylalkyl group substituted with 1 to 4 hydrophilic groups selected from hydroxy, alkoxy, —C(O)OR⁵, —SOR⁶, —SO₂R⁶, nitro, amido, ureido, carbamato, —SR⁷, —NR⁸R⁹, or poly-alkyleneoxide;
X¹ and X² are independently oxygen or sulfur;
R¹, R², R³, R⁴, R⁵, R⁶, R⁷, R⁸, and R⁹ are independently selected from hydrogen and C₁ to C₄ hydrocarbyl;
r and s are independently 0 or an integer from 1 to 20;
a, b, c, d, and e independently represent an integer from 1 to 4; and
M is either two hydrogen ions, a single monovalent metal ion, two monovalent metal ions, a divalent metal ion, a trivalent metal ion, a tetravalent metal ion, a pentavalent metal ion, a hexavalent metal ion wherein the porphyrin-metal complex derived from a single monovalent metal ion is charge-balanced by a counter cation, and the porphyrin-metal complex derived from a trivalent, tetravalent, pentavalent, hexavalent metal ion is charge-balanced by an appropriate number of counter anions, dianions, or trianions;
b) the compound of formula a) in which:
i. a, b, c, and dare 1;
ii. two of Y¹, Y², Y³ and Y⁴ are represented by formula (2);
iii. X¹ is oxygen, R¹ and R² are hydrogen, and r is 1;
iv. Z is selected from the carborane clusters —C₂HB₉H₁₀ or —C₂HB₁₀H₁₀, wherein —C₂HB₉H₁₀ is nido ortho-, meta-, or para-carborane, and —C₂HB₁₀H₁₀ is closo ortho-, meta-, or para-carborane;
v. the substituents represented by formula (2) are in the trans configuration;
vii. the substituents represented by formula (2) are on the meta position of each phenyl ring;
viii. the two Y¹-Y⁴ not represented by formula (2) are selected from the group consisting of D or L, alpha and beta forms of glucose and galactose; and
ix. M is vanadium, manganese, iron; ruthenium, technetium, chromium, platinum, cobalt, nickel, copper, zinc, germanium, indium, tin, yttrium, gold, barium, tungsten, gadolinium, or combination thereof; and
c) the compound of formula a) in which:
i. a, b, c, and dare 1;
ii. two of Y¹, Y², Y³ and Y⁴ are represented by formula (2);
iii. Z is represented by formula (3);
iv. D is selected from the carborane clusters —C₂HB₉H₁₀ or —C₂HB₁₀H₁₀, wherein —C₂HB₉H₁₀ is nido ortho-, meta-, or para-carborane, and —C₂HB₁₀H₁₀ is closo ortho-, meta-, or para-carborane;
iv. the structures —X²—(CR³R⁴)ₛ-D are in the 3 and 5 positions of each phenyl ring;
V. X¹ and X² are oxygen, R¹, R², R³ and R⁴ are hydrogen, r and s are 1, and e is 2;
vi. the substituents represented by formula (2) are in the trans configuration;
vii. the two Y¹-Y⁴ not represented by formula (2) are selected from the group consisting of D or L, alpha and beta forms of glucose and galactose; and
viii. M is vanadium, manganese, iron, ruthenium, technetium, chromium, platinum, cobalt, nickel, copper, zinc, germanium, indium, tin, yttrium, gold, barium, tungsten, gadolinium, or combination thereof;
and the imaging said subject.

23. The method according to claim 22 wherein said imaging is by a method selected from magnetic resonance imaging (MRI), single photon emission computed tomography (SPECT), or positron emission tomography (PET) methods.

24. The method according 23 wherein imaging by single photon emission computed tomography (SPECT) or positron emission tomography (PET) utilizes a SPECT- and/or PET-imageable radioactive metal ion (M).

25. The method according to claim 22 wherein said imaging utilizes magnetic resonance imaging (MRI) wherein M is a paramagnetic metal ion.

26. A method of bimodal cancer treatment in a subject comprising: administering to the subject a composition comprising a compound selected from the group consisting of:
a) a compound of the formula 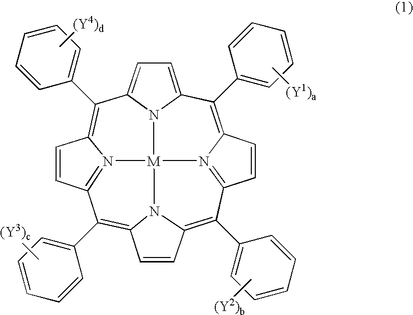
wherein:
at least one of Y¹, Y², Y³, and Y⁴ represents a mono-saccharide, di-saccharide, tri-saccharide, or oligo-saccharide and at least one of Y¹, Y², Y³, and Y⁴ is a substituent represented by formula (2)
—X¹—(CR¹R²)ᵣ-Z (2)
wherein Z is a carborane cluster or a substituent represented by formula (3): 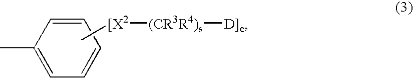
wherein D is a carborane cluster;
Y¹, Y², Y³, and Y⁴ are independently on either or both of the ortho and/or meta positions or on the para position on the phenyl rings;
the Y¹, Y², Y³, and Y⁴ that are not a mono-saccharide, di-saccharide, tri-saccharide, or oligo-saccharide and that are not represented by formula (2) or (3) are independently hydrogen, a hydrocarbyl, non-aromatic carbocyclic, non-aromatic heterocyclic, aryl, alkylaryl, or arylalkyl group substituted with 1 to 4 hydrophilic groups selected from hydroxy, alkoxy, —C(O)OR⁵, —SOR⁶, —SO₂R⁶, nitro, amido, ureido, carbamato, —SR⁷, —NR⁸R⁹, or poly-alkyleneoxide;
X¹ and X² are independently oxygen or sulfur;
R¹, R², R³, R⁴, R⁵, R⁶, R⁷, R⁸, and R⁹ are independently selected from hydrogen and C₁ to C₄ hydrocarbyl;
r and s are independently 0 or an integer from 1 to 20;
a, b, c, d, and e independently represent an integer from 1 to 4; and
M is either two hydrogen ions, a single monovalent metal ion, two monovalent metal ions, a divalent metal ion, a trivalent metal ion, a tetravalent metal ion, a pentavalent metal ion, a hexavalent metal ion wherein the porphyrin-metal complex derived from a single monovalent metal ion is charge-balanced by a counter cation, and the porphyrin-metal complex derived from a trivalent, tetravalent, pentavalent, hexavalent metal ion is charge-balanced by an appropriate number of counter anions, dianions, or trianions;
b) the compound of formula a) in which:
i. a, b, c, and d are 1;
ii. two of Y¹, Y², Y³ and Y⁴ are represented by formula (2);
iii. X¹ is oxygen, R¹ and R² are hydrogen, and r is 1;
iv. Z is selected from the carborane clusters —C₂HB₉H₁₀ or —C₂HB₁₀H₁₀, wherein —C₂HB₉H₁₀ is nido ortho-, meta-, or para-carborane, and —C₂HB₁₀H₁₀ is closo ortho-, meta-, or para-carborane;
v. the substituents represented by formula (2) are in the trans configuration;
vii. the substituents represented by formula (2) are on the meta position of each phenyl ring;
viii. the two Y¹-Y⁴ not represented by formula (2) are selected from the group consisting of D or L, alpha and beta forms of glucose and galactose; and
ix. M is vanadium, manganese, iron, ruthenium, technetium, chromium, platinum, cobalt, nickel, copper, zinc, germanium, indium, tin, yttrium, gold, barium, tungsten, gadolinium, or combination thereof; and
c) the compound of formula a) in which:
i. a, b, c, and d are 1;
ii. two of Y¹, Y², Y³ and Y⁴ are represented by formula (2);
iii. Z is represented by formula (3);
iv. D is selected from the carborane clusters —C₂HB₉H₁₀ or —C₂HB₁₀H₁₀, wherein —C₂HB₉H₁₀ is nido ortho-, meta-, or para-carborane, and —C₂HB₁₀H₁₀ is closo ortho-, meta-, or para-carborane;
iv. the structures —X²—(CR³R⁴)ₛ-D are in the 3 and 5 positions of each phenyl ring;
v. X¹ and X² are oxygen, R¹, R², R³ and R⁴ are hydrogen, r and s are 1, and e is 2;
vi. the substituents represented by formula (2) are in the trans configuration;
vii. the two Y¹-Y⁴ not represented by formula (2) are selected from the group consisting of D or L, alpha and beta forms of glucose and galactose; and
viii. M is vanadium, manganese, iron, ruthenium, technetium, 70 chromium, platinum, cobalt, nickel, copper, zinc, germanium, indium, tin, yttrium, gold, barium, tungsten, gadolinium, or combination thereof;
and irradiating said subject.

27. The method according to claim 26 wherein said irradiation is by a method utilizing thermal or epithermal neutrons, or laser red light.

28. The method according to claim 27 wherein said bimodal cancer treatment comprises boron neutron capture therapy (BNCT).

29. The method according claim 27 wherein said bimodal cancer treatment comprises photodynamic therapy (PDT).

30. The method according to claim 26 wherein said bimodal cancer treatment is augmented by imaging.

31. The method according to claim 30 wherein imaging is by single photon emission computed tomography (SPECT) or positron emission tomography (PET) wherein M is a SPECT- and/or PET-imageable radioactive metal ion.

32. The method according to claim 30 wherein said imaging utilizes magnetic resonance imaging (MRI) wherein M is a paramagnetic metal ion.

33. A compound of the formula 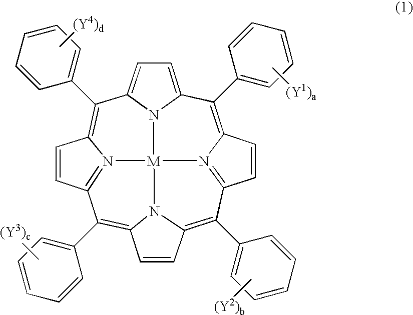
wherein:
at least one of Y¹, Y², Y³, and Y⁴ represents a mono-saccharide, di-saccharide, tri-saccharide, or oligo-saccharide and at least one of Y¹, Y², Y³, and Y⁴ is a substituent represented by formula (2)
—X¹—(CR¹R²)ᵣ-Z (2)
wherein Z is a carborane cluster or a substituent represented by formula (3): 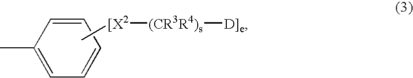
where D is a carborane cluster;
Y¹, Y², Y³, and Y⁴ are independently on either or both of the ortho and/or meta positions or on the para position on the phenyl rings;
the Y¹, Y², Y³, and Y⁴ that are not a mono-saccharide, di-saccharide, tri-saccharide, or oligo-saccharide and that are not represented by formula (2) or (3) are independently hydrogen, a hydrocarbyl, non-aromatic carbocyclic, non-aromatic heterocyclic, aryl, alkylaryl, or arylalkyl group substituted with 1 to 4 hydrophilic groups selected from hydroxy, alkoxy, —C(O)OR⁵, —SOR⁶, —SO₂R⁶, nitro, amido, ureido, carbamato, —SR⁷, —NR⁸R⁹, or poly-alkyleneoxide;
Z and D are carborane clusters comprising at least two carbon atoms and at least three boron atoms, or at least one carbon atom and at least five boron atoms, within a cage structure;
X¹ and X² are independently oxygen or sulfur;
R¹, R², R³, R⁴, R⁵, R⁶, R⁷, R⁸, and R⁹ are independently selected from hydrogen and C₁ to C₄ hydrocarbyl;
r and s are independently 0 or an integer from 1 to 20;
a, b, c, d, and e independently represent an integer from 1 to 4; and
M is a trivalent, tetravalent, pentavalent, or hexavalent metal ion; and
wherein the porphyrin-metal complex is charge-balanced by one or more porphyrin compounds containing a divalent negative charge.
